(19) United States
(12) Patent Application Publication
(10) Pub. No.: US 2024/0390367 A1
(43) Pub. Date: Nov. 28, 2024

(54) METHODS OF TREATING VITILIGO WITH UPADACITINIB

(71) Applicant: AbbVie Inc., North Chicago, IL (US)

(72) Inventors: Sumit Bhatnagar, Highland Park, IL (US); Mohamed-Eslam F. Mohamed, Gurnee, IL (US); Heidi S. Camp, Winnetka, IL (US)

(21) Appl. No.: 18/671,573

(22) Filed: May 22, 2024

Related U.S. Application Data
(60) Provisional application No. 63/543,713, filed on Oct. 11, 2023.
(60) Provisional application No. 63/468,465, filed on May 23, 2023.

(51) Int. Cl. A61K 31/4985; A61K 9/00; A61K 9/08; A61K 47/12; A61P 17/00

(52) U.S. Cl. CPC A61K 31/4985; A61K 9/0053; A61K 9/08; A61K 47/12; A61P 17/00

(57) ABSTRACT

The present disclosure is directed to methods for treating vitiligo using the selective JAK1 inhibitor upadacitinib.

### CROSS-REFERENCE TO RELATED APPLICATIONS

This application claims priority to U.S. Provisional Application No. 63/468,465, filed May 23, 2023, and U.S. Provisional Application No. 63/543,713, filed Oct. 11, 2023, each of which is herein incorporated by reference in its entirety.

### FIELD OF THE DISCLOSURE

The present disclosure is directed to methods for treating vitiligo with the JAK1 selective inhibitor upadacitinib.

### BACKGROUND OF THE DISCLOSURE

Upadacitinib is a JAK1 selective inhibitor approved for the treatment of rheumatoid arthritis, psoriatic arthritis, ankylosing spondylitis, non-radiographic spondyloarthritis, ulcerative colitis in adults, and atopic dermatitis.

Vitiligo is a chronic autoimmune disorder that affects 0.5% to 2.0% of the population. It is characterized by skin depigmentation caused by the loss of melanocytes (Bergqvist et al., Dermatology 2020, 236:571-92; Ezzedine et al., Pigment Cell Melanoma Res 2012, 25:E1-13). Vitiligo is classified into two major forms (nonsegmental or segmental) based on the distribution of lesions (Taieb et al., Pigment Cell Res 2007, 20:27-35). Studies have shown that vitiligo places a substantial burden on the patients' quality of life. Patients with vitiligo incur direct costs associated with medical treatment as well as indirect costs in the form of psychosocial effects, loss of work productivity, and lost social and career opportunities. Accordingly, there is a substantial unmet need for efficacious and cost-effective treatments.

Treatment of vitiligo remains one of the most difficult dermatologic challenges. Currently, there is no cure available. Few treatment options exist and are generally limited to topical corticosteroids, ultraviolet B irradiation, and surgical management (Bergqvist et al., 2020). For reasons including patient convenience and compliance, particularly in long-term treatment, it would be desirable to provide an efficacious oral therapeutic for the treatment of vitiligo.

### SUMMARY OF THE DISCLOSURE

The present disclosure provides methods for treating vitiligo with the selective JAK1 inhibitor, upadacitinib. The treatment methods generally comprise administering to a patient in need thereof a therapeutically effective amount of upadacitinib.

In one aspect is provided a method of treating an adult human patient having vitiligo, the method comprising orally administering once daily to the patient 15 mg of upadacitinib.

In some embodiments, the patient has non-segmental vitiligo (NSV).

In some embodiments, the NSV is stable.

In some embodiments, the NSV is active.

In some embodiments, the patient has, before initiating the treatment, a Facial Vitiligo Area Scoring Index (F-VASI) of ≥0.5.

In some embodiments, the patient has, before initiating the treatment, a Total Vitiligo Area Scoring Index (T-VASI)≥5.

In some embodiments, the patient has not received any immunomodulatory biologic therapy prior to initiating the treatment.

In some embodiments, the patient has not received any topical JAK inhibitor within 12 weeks of initiating treatment.

In some embodiments, the patient has leukotrichia in ≤33% of the affected areas.

In some embodiments, the patient achieves F-VASI 75 at 24 weeks after initiating the treatment.

In some embodiments, the patient achieves at 36 weeks after initiating the treatment one or more of a T-VASI 50, a T-VASI 75, a T-VASI 90, a F-VASI 50, or a F-VASI 90.

In some embodiments, the patient achieves T-VASI 50 at 48 weeks after the first daily administration.

In some embodiments, the patient achieves F-VASI 75 at 48 weeks after the first daily administration.

In some embodiments, the patient achieves T-VASI 50 at 52 weeks after the first daily administration.

In some embodiments, the patient achieves F-VASI 75 at 52 weeks after the first daily administration.

In some embodiments, the patient achieves an improvement in Global Impression of Change-Overall Vitiligo (PaGIC-V) at 36 weeks after initiating the treatment.

In some embodiments, the patient achieves a vitiligo noticeability scale (VNS) score of “A lot less noticeable (4)” or “No longer noticeable (5)” at 36 weeks after initiating the treatment.

In some embodiments, the patient achieves an improvement from baseline in the vitiligo extent score (VES) at 36 weeks after initiating the treatment.

In some embodiments, the patient achieves an improvement from baseline in the VitiQoL at 36 weeks after initiating the treatment.

In some embodiments, the patient achieves a Dermatology Life Quality Index (DLQI) total score of “0” or “1” at 36 weeks after initiating the treatment.

In some embodiments, the patient achieves an improvement from baseline in the Hospital Anxiety and Depression Scale (HADS) scores at 36 weeks after initiating the treatment.

In some embodiments, the patient achieves an improvement in time to onset of repigmentation relative to a patient which has not been treated with upadacitinib.

In some embodiments, the patient achieves a Physician's Global Impression of Change-Vitiligo (PhGIC-V) score of “Much better (1)” or “A little better (2)” at 36 weeks after initiating the treatment. In some embodiments, the patient achieves a Patient's Global Impression of Change-Vitiligo (PaGIC-V) score of “Much better (1)” or “A little better (2)” at 36 weeks after initiating the treatment. In some embodiments, the patient achieves a Face-Physician Global Vitiligo Assessment (F-PhGVA) score of “No depigmentation (0)” or “Limited extent of depigmentation (1)” at 36 weeks after initiating the treatment. In some embodiments, the patient achieves a Total-Physician Global Vitiligo Assessment (T-PhGVA) score of “No depigmentation (0)” or “Limited extent of depigmentation (1)” at 36 weeks after initiating the treatment. In some embodiments, the patient achieves a Face-Patient Global Vitiligo Assessment (F-PaGVA) score of “No depigmentation (0)” or “Limited extent of depigmentation (1)” at 36 weeks after initiating the treatment. In some embodiments, the patient achieves a Total-Patient Global Vitiligo Assessment (T-PaGVA) score of “No depigmentation (0)” or “Limited extent of depigmentation (1)” at 36 weeks after initiating the treatment.

In some embodiments, the patient achieves an improvement in itching or burning sensation within 36 weeks of initiating the treatment.

In some embodiments, the method halts the progression of vitiligo within 36 weeks of initiating the treatment.

In some embodiments, the method results in a reduction from baseline in one or more biomarkers of vitiligo within 36 weeks of initiating the treatment. In some embodiments, the biomarker is an inflammatory chemotactic protein or a T-cell activation marker. In some embodiments, the biomarker is CXCL9, CXCL10, IL12B, TNFSF9, or a combination thereof.

In some embodiments, the patient is concomitantly treated with exposure to ultraviolet light.

In another aspect is provided a method of treating vitiligo in a human pediatric patient, the method comprising administering a therapeutically effective amount of upadacitinib to the pediatric patient, wherein:
if the pediatric patient has a body weight in a range from about 10 kg to less than about 20 kg:
   (i) the upadacitinib is administered twice daily at a dose of 3 mg each (3 mg BID), or
   (ii) the upadacitinib is administered twice daily at a dose of 6 mg each (6 mg BID);
if the pediatric patient has a body weight in a range from about 20 kg to less than about 30 kg:
   (i) the upadacitinib is administered twice daily at a dose of 4 mg each (4 mg BID), or
   (ii) the upadacitinib is administered twice daily at a dose of 8 mg each (8 mg BID); and
if the pediatric patient has a body weight of about 30 kg or greater:
   (i) the upadacitinib is administered twice daily at a dose of 6 mg each (6 mg BID),
   (ii) the upadacitinib is administered twice daily at a dose of 8 mg each (8 mg BID), or
   (ii) the upadacitinib is administered once daily at a dose of 15 mg (15 mg QD).

In some embodiments, the twice daily at a dose of 3 mg of upadacitinib, the twice daily at a dose of 4 mg of upadacitinib, the twice daily at a dose of 6 mg of upadacitinib or the twice daily at a dose of 8 mg of upadacitinib is administered to the pediatric patient as a stable oral pharmaceutical solution.

In some embodiments, the stable oral pharmaceutical solution comprises upadacitinib, a buffer and/or pH adjusting agent, a preservative, a sweetener, and water.

In some embodiments, the oral solution comprises upadacitinib at a concentration of about 0.5 mg/mL or about 1 mg/mL.

In some embodiments, the once daily at a dose of 15 mg of upadacitinib is administered to the pediatric patient as an extended-release tablet.

In some embodiments, the pediatric patient has non-segmental vitiligo (NSV).

In some embodiments, the NSV is stable.

In some embodiments, the NSV is active.

In some embodiments, the pediatric patient has, before initiating the treatment, a Facial Vitiligo Area Scoring Index (F-VASI) of ≥0.5.

In some embodiments, the pediatric patient has, before initiating the treatment, a Total Vitiligo Area Scoring Index (T-VASI)≥5.

In some embodiments, the pediatric patient has not received any immunomodulatory biologic therapy prior to initiating treatment.

In some embodiments, the pediatric patient has not received any topical JAK inhibitor within 12 weeks of initiating treatment.

In some embodiments, the pediatric patient has leukotrichia in ≤33% of the affected areas.

In some embodiments, the pediatric patient achieves a F-VASI 75 at 24 weeks after initiating the treatment.

In some embodiments, the pediatric patient achieves at 36 weeks after initiating the treatment one or more of a T-VASI 50, a T-VASI 75, a T-VASI 90, a F-VASI 50, or a F-VASI 90.

In some embodiments, the patient achieves T-VASI 50 at 48 weeks after the first daily administration.

In some embodiments, the pediatric patient achieves F-VASI 75 at 48 weeks after the first daily administration.

In some embodiments, the pediatric patient achieves T-VASI 50 at 52 weeks after the first daily administration.

In some embodiments, the pediatric patient achieves F-VASI 75 at 52 weeks after the first daily administration.

In some embodiments, the pediatric patient achieves an improvement in the Global Impression of Change-Overall Vitiligo (PaGIC-V) at 36 weeks after initiating the treatment.

In some embodiments, the pediatric patient achieves a vitiligo noticeability scale (VNS) score of “A lot less noticeable (4)” or “No longer noticeable (5)” at 36 weeks after initiating the treatment.

In some embodiments, the pediatric patient achieves an improvement from baseline in the vitiligo extent score (VES) at 36 weeks after initiating the treatment.

In some embodiments, the pediatric patient achieves an improvement from baseline in the VitiQoL at 36 weeks after initiating the treatment.

In some embodiments, the pediatric patient achieves a Dermatology Life Quality Index (DLQI) total score of “0” or “1” at 36 weeks after initiating the treatment.

In some embodiments, the pediatric patient achieves an improvement from baseline in the Hospital Anxiety and Depression Scale (HADS) scores at 36 weeks after initiating the treatment.

In some embodiments, the pediatric patient achieves an improvement in time to onset of repigmentation relative to a pediatric patient which has not been treated with upadacitinib.

In some embodiments, the pediatric patient achieves an improvement in itching or burning sensation within 36 weeks of initiating the treatment.

In some embodiments, the pediatric patient achieves a Physician's Global Impression of Change-Vitiligo (PhGIC-V) score of “Much better (1)” or “A little better (2)” at 36 weeks after initiating the treatment. In some embodiments, the pediatric patient achieves a Patient's Global Impression of Change-Vitiligo (PaGIC-V) score of “Much better (1)” or “A little better (2)” at 36 weeks after initiating the treatment. In some embodiments, the pediatric patient achieves a Face-Physician Global Vitiligo Assessment (F-PhGVA) score of “No depigmentation (0)” or “Limited extent of depigmentation (1)” at 36 weeks after initiating the treatment. In some embodiments, the pediatric patient achieves a Total-Physician Global Vitiligo Assessment (T-PhGVA) score of “No depigmentation (0)” or “Limited extent of depigmentation (1)” at 36 weeks after initiating the treatment. In some embodiments, the pediatric patient achieves a Face-Patient Global Vitiligo Assessment (F-PaGVA) score of “No depigmentation (0)” or “Limited extent of depigmentation (1)” at 36 weeks after initiating the treatment. In some embodiments, the pediatric patient achieves a Total-Patient Global Vitiligo Assessment (T-PaGVA) score of “No depigmentation (0)” or “Limited extent of depigmentation (1)” at 36 weeks after initiating the treatment.

In some embodiments, the method halts the progression of vitiligo within 36 weeks of initiating the treatment.

In some embodiments, the method results in a reduction from baseline in one or more biomarkers of vitiligo within 36 weeks of initiating the treatment. In some embodiments, the biomarker is an inflammatory chemotactic protein or a T-cell activation marker. In some embodiments, the biomarker is CXCL9, CXCL10, IL12B, TNFSF9, or a combination thereof.

In some embodiments, the pediatric patient is concomitantly treated with exposure to ultraviolet light.

### BRIEF DESCRIPTION OF THE DRAWINGS

FIG. 1 is a schematic illustration of a clinical study according to a non-limiting embodiment of the disclosure, as described in Example 1.

FIG. 2 is a graphical depiction of efficacy according to the primary endpoint (percent change from baseline in F-VASI at Week 24 (MMRM)) for subjects in the clinical study of Example 1.

FIG. 3 is a graphical depiction of efficacy according to the secondary endpoint (proportion achieving F-VASI 75 at Week 24 (NRI)) for subjects in the clinical study of Example 1.

FIG. 4 is a graphical depiction of efficacy according to the secondary endpoint (percent change from baseline in T-VASI at Week 24 (NRI)) for subjects in the clinical study of Example 1.

FIG. 5A is a graphical depiction of efficacy according to a secondary endpoint (percent change from baseline in T-VASI 50) at weeks 24 and 52 for subjects in the clinical study of Example 1.

FIG. 5B is a graphical depiction of efficacy according to a secondary endpoint (percent change from baseline in F-VASI 75) at weeks 24 and 52 for subjects in the clinical study of Example 1.

FIG. 6A is a graphical depiction of model-predicted and observed exposure-response efficacy analyses for endpoints CFB F-VASI and CFB T-VASI at week 24 according to Example 2.

FIG. 6B is a graphical depiction of model-predicted and observed exposure-response efficacy analyses for endpoints F-VASI 75 and T-VASI 50 at week 24 according to Example 2.

FIG. 7A is a graphical depiction of model-predicted efficacy response rates for the endpoint F-VASI 75 for various doses of upadacitinib at week 24 according to Example 2.

FIG. 7B is a graphical depiction of model-predicted efficacy response rates for the endpoint T-VASI 50 for various doses of upadacitinib at week 24 according to Example 2.

FIG. 8A is a graphical depiction of exploratory exposure-response analyses for efficacy endpoint CFB F-VASI at week 52 according to Example 2.

FIG. 8B is a graphical depiction of exploratory exposure-response analyses for efficacy endpoint CFB T-VASI at week 52 according to Example 2.

FIG. 9A is a graphical depiction of model-predicted response rates for the endpoint F-VASI 75 for a 15 mg dose of upadacitinib according to Example 2.

FIG. 9B is a graphical depiction of model-predicted response rates for the endpoint T-VASI 50 for a 15 mg dose of upadacitinib according to Example 2.

FIG. 10A is a graphical depiction of % change facial vitiligo area by cherry 3D imaging for various doses of upadacitinib and placebo according to Example 3.

FIG. 10B is a graphical depiction of % change in F-VASI for various doses of upadacitinib and placebo according to Example 3.

FIG. 11 is a schematic illustration of a clinical study according to a non-limiting embodiment of the disclosure, as described in Example 4.

FIG. 12 is a graphical depiction of percent change from baseline in F-VASI through Week 52 according to Example 7.

FIG. 13 is a graphical depiction of percent change from baseline in T-VASI through Week 52 according to Example 7.

FIG. 14A is a graphical depiction of response rate for achievement of F-VASI 50 at Week 52 according to Example 7.

FIG. 14B is a graphical depiction of response rate for achievement of F-VASI 75 at Week 52 according to Example 7.

FIG. 14C is a graphical depiction of response rate for achievement of T-VASI 50 at Week 52 according to Example 7.

FIG. 15A is a graphical depiction of response rate for achievement of F-VASI 75 at Week 24 according to Example 7.

FIG. 15B is a graphical depiction of response rate for achievement of F-VASI 75 at Week 36 according to Example 7.

FIG. 15C is a graphical depiction of response rate for achievement of F-VASI 75 at Week 52 (non-responder imputation while incorporating multiple imputation; NRI-MI) according to Example 7.

FIG. 15D is a graphical depiction of response rate for achievement of F-VASI 75 at Week 52 (AO) according to Example 7.

FIG. 16A is a graphical depiction of response rate for achievement of T-VASI 50 at Week 24 according to Example 7.

FIG. 16B is a graphical depiction of response rate for achievement of T-VASI 50 at Week 36 according to Example 7.

FIG. 16C is a graphical depiction of response rate for achievement of T-VASI 50 at Week 52 (NRI-MI) according to Example 7.

FIG. 16D is a graphical depiction of response rate for achievement of T-VASI 50 at Week 52 (as observed; AO) according to Example 7.

### DETAILED DESCRIPTION OF THE DISCLOSURE

### I. Definitions

Section headings as used in this section and the entire disclosure are not intended to be limiting.

Where a numeric range is recited, each intervening number within the range is explicitly contemplated with the same degree of precision. For example, for the range 6 to 9, the numbers 7 and 8 are contemplated in addition to 6 and 9, and for the range 6.0 to 7.0, the numbers 6.0, 6.1, 6.2, 6.3, 6.4, 6.5, 6.6, 6.7, 6.8, 6.9 and 7.0 are explicitly contemplated. In the same manner, all recited ratios also include all sub-ratios falling within the broader ratio.

The singular forms “a,” “an” and “the” include plural referents unless the context clearly dictates otherwise.

The term “about” generally refers to a range of numbers that one of skill in the art would consider equivalent to the recited value (i.e., having the same function or result). In many instances, the term “about” may include numbers that are rounded to the nearest significant figure.

Unless the context requires otherwise, the terms “comprise,” “comprises,” and “comprising” are used on the basis and clear understanding that they are to be interpreted inclusively, rather than exclusively, and that Applicant intends each of those words to be so interpreted in construing this patent, including the claims below.

The term “AUC” refers to the area under the curve. AUC is the definite integral of a curve that describes the variation of a drug concentration in blood plasma as a function of time.

The term “Cₘₐₓ” refers to the plasma concentration of the referent drug at Tₘₐₓ, expressed herein as ng/mL, produced by the oral ingestion of a single dose, or indicated number of doses, of the dosage form or pharmaceutical composition, such as the dosage forms and compositions of the present disclosure. Unless specifically indicated, Cₘₐₓ refers to the overall maximum observed concentration.

The terms “treating”, “treatment”, and “therapy” and the like, as used herein, are meant to include therapeutic measures for a disease or disorder leading to a clinically desirable or beneficial effect, including but not limited to clinically significant improvement in depigmentation over a period of time.

As used herein, the term “pediatric patient” refers to a human patient of less than 18 years old. The terms “patient” and “subject” are used interchangeably herein.

“Pharmaceutically acceptable salts” refers to those salts which retain the biological effectiveness and properties of the free bases and which are obtained by reaction with inorganic acids, for example, hydrochloric acid, hydrobromic acid, sulfuric acid, nitric acid, and phosphoric acid or organic acids such as sulfonic acid, carboxylic acid, organic phosphoric acid, methanesulfonic acid, ethanesulfonic acid, p-toluenesulfonic acid, citric acid, fumaric acid, maleic acid, succinic acid, benzoic acid, salicylic acid, lactic acid, mono-malic acid, mono oxalic acid, tartaric acid such as mono tartaric acid (e.g., (+) or (−)-tartaric acid or mixtures thereof), amino acids (e.g., (+) or (−)-amino acids or mixtures thereof), and the like. These salts can be prepared by methods known to those skilled in the art. Examples of pharmaceutically acceptable salts of upadacitinib may be found in WO 2017/066775, which is hereby incorporated by reference in its entirety.

The term “active vitiligo” as used herein refers to vitiligo showing new lesions or progression (enlargement) of existing lesions within the last 6 months; and/or the presentation of clinical subtypes indicative of progressing vitiligo (Confetti-like depigmentation, Trichrome pattern, Koebner phenomenon).

The term “stable vitiligo” as used herein refers to vitiligo with no new lesions and/or no extension of pre-existing lesions.

The Vitiligo Area Scoring Index (VASI) is a qualitative tool analogous to the Psoriasis Area and Severity Index (PASI). VASI is used to evaluate the severity of vitiligo and/or treatment efficacy. It is the current gold-standard for semi-objective assessment of vitiligo and represents a composite estimate of overall area of vitiligo patches and degree of depigmentation within these patches. The VASI is determined by approximating the percent body surface area affected, which is multiplied by the percent degree of depigmentation in those areas. In determining the body surface area percent, the hand is equal to 1%, and the thumb is equal to 0.1%. VASI may be referenced as total (T-VASI) or facial (F-VASI). The F-VASI includes contributions from the face, with a possible range from 0 to 3. For the purpose of assessing F-VASI, the area of the face includes the forehead to the original hairline, temple, around the eyes (including eyelids), nose, check (mid and lateral), and around the mouth (including the chin). The ‘face’ excludes the lips, cars, and scalp

The Vitiligo Noticeability Scale (VNS) is a single-item, validated questionnaire used in clinical trials to assess the noticeability of vitiligo lesions following therapy.1 The item is scored on a 5-point scale: 1=more noticeable; 2=as noticeable; 3=slightly less noticeable; 4=a lot less noticeable; and 5=no longer noticeable. A score of 4 or 5 represents treatment success.

The Vitiligo Quality-of-Life (VitiQoL) is a validated questionnaire used in clinical trials to assess stigma-related vitiligo impacts. The VitiQoL uses subject-elicited social, affective, and behavior items, asking the subject's appraisal of the vitiligo-related impacts over the last month. Fifteen items are scored on a 7-point scale ranging from 0 (“Not at all”) to 6 (“All of the time”). Item scores (0 to 6) are summed to provide a total score range of 0 to 90; higher scores indicate greater impairment of quality of life (QoL). An additional item, not contributing to the total impact score, asks the patient to rank the current severity of their condition on a 7-point scale ranging from 0 (“No skin involvement”) to 6 (“Most severe case”). The VitiQoL will be administered on the tablet at site visits according to the PRO assessment schedule as outlined in the protocol study activities table.

The Dermatology Life Quality Index (DLQI) is a 10-item questionnaire developed and validated for use in measuring QOL in patients with skin conditions.3 It has been used in clinical practice and a number of clinical trials to assess the impact of vitiligo disease symptoms and treatment on QoL.4 It consists of 10 questions assessing impact of skin diseases on different aspects of subject's QoL over the prior week. The DLQI measures several domains, including symptoms and feelings, daily activities, leisure, work or school, personal relationships, and treatment burden. Each item is scored on a 4-point scale: 0=not at all/not relevant; 1=a little; 2=a lot; and 3=very much. Item scores (0 to 3) are added to provide a total score range of 0 to 30; higher scores indicate greater impairment of QoL. For general inflammatory skin conditions, a change in DLQI score of at least 4 points is considered the minimum clinically important difference.

The Hospital Anxiety and Depression Scale (HADS) is a 14-item questionnaire, with 7 items related to anxiety (HADS-A) and seven items related to depression (HADS-D). Each item is scored from 0 to 3; scores for each subscale range from 0 to 21 and scores for the entire scale (emotional distress) range from 0 to 42, with higher scores indicating more distress. For each domain, scores 7 or lower are considered normal, 8 to 10 are borderline, and 11 or higher indicate clinical anxiety or depression. HADS will be administered on the tablet at site visits according to the PRO assessment schedule as outlined in the protocol study activities table.

The Patient's Global Impression of Change-Vitiligo (PaGIC-V) is a survey asking subjects to rate the overall change in their vitiligo symptoms by comparing the severity of their vitiligo symptoms right now with the severity of their vitiligo symptoms since they started the study treatment. Responses range from 1=“Much better” to 5=“Much worse.”

The Total-Patient Global Vitiligo Assessment (T-PaGVA) and Face-Patient Global Vitiligo Assessment (F-PaGVA) are two region-specific patient global assessments of severity will measure the subject's perceptions of their current vitiligo condition. Both are single-item questionnaires with 5-point categorical response options. The T-PaGVA measures the subject's perception of their vitiligo for all body regions. The F-PaGVA measures the subject's perception of their facial vitiligo. Each scale is constructed to ask about severity of disease based upon the extent of area covered by depigmented skin. Response options range from 1=no depigmentation to 5=very extensive depigmentation.

The Vitiligo Extent Score (VES) is a clinical scoring tool to measure the overall vitiligo involvement of the body (extent). The VES tool is clinical picture-based), involving 19 separate areas of the body, reflecting 6 degrees of involvement (Grade 0-6: involved per region 0%, 1%, 5%, 10%, 25%, 50%, and 75%, respectively).

The Total-Physician Global Vitiligo Assessment (T-PhGVA) and Face-Physician Global Vitiligo Assessment (F-PhGVA) are tools to quantify the severity of vitiligo. They use a 5-point scale: 0=no depigmentation; 1=limited extent of depigmentation; 2=moderate extent of depigmentation; 3=extensive depigmentation; and 4=very extensive depigmentation. The T-PhGVA includes contributions from all body regions, while the F-PhGVA includes contributions from the face.

The Physician's Global Impression of Change-Vitiligo (PhGIC-V) is a survey in which the investigator rates the overall change in the subject's vitiligo symptoms by comparing the severity of vitiligo symptoms right now with the severity of vitiligo symptoms since the subject started the study treatment. Responses range from 1=“Much better” to 5=“Much worse.”

The Individual Component Vitiligo Assessment (ICVA) is a paper-based worksheet completed by the investigator and is used to collect exploratory information regarding vitiligo involvement on the genitals and individual components of the face using the following 2 assessments: PhGVA and how much vitiligo lesions are bothersome to subjects. Level of bother is based upon a question asked of the subject about how much their vitiligo on the genitals and individual areas of the face bothers them and uses a 4-point scale: 1=not at all bothersome; 2=slightly bothersome; 3=moderately bothersome; and 4=extremely bothersome. There is also a “N/A” (not applicable) response option for areas that are not affected by vitiligo. Individual components of the face include the forehead/temple, around the eyes, nose, middle cheek (medially to the lateral canthus of the eye), lateral cheek (laterally to the lateral canthus of the eye), around the mouth, lips, ears, and scalp.

### II. JAK1 Inhibition

Upadacitinib (3S,4R)-3-ethyl-4-(3H-imidazo[1,2-a]pyrrolo[2,3-e]pyrazin-8-yl)-N-(2,2,2-trifluoroethyl) pyrrolidine-1-carboxamide), or a pharmaceutically acceptable salt or solid state form thereof, is an oral Janus kinase (JAK) inhibitor that displays unique selectivity for the JAK1 receptor. Upadacitinib has the structure shown below:

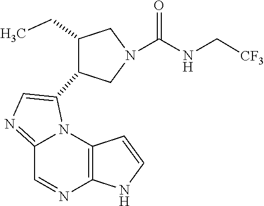 [SMILES: CC[C@@H]1CN(C(=O)NCC(F)(F)F)C[C@@H]1c1cnc2cnc3[nH]ccc3n12]

The dosage strength of upadacitinib recited in the present application is based on the weight of anhydrous freebase upadacitinib present in the active ingredient delivered to the patient. For example, a dose of “15 mg of upadacitinib” or “UPA 15 MG” refers to the 15 mg amount of the neutral upadacitinib freebase present in the active ingredient, not including any coformer (e.g., solvent or water molecule(s)) of a solvate or hydrate (including hemihydrate) or counteranions of a pharmaceutically acceptable salt), that may also be present in the active ingredient. So, for example, the administration of “15 mg of upadacitinib” includes the administration of 15.4 mg of crystalline upadacitinib freebase hemihydrate (which includes ½ of a water conformer molecule per upadacitinib freebase molecule) which delivers 15 mg of anhydrous freebase upadacitinib to a patient.

### III. Treatment of Vitiligo

Provided herein are methods of treating vitiligo with the selective JAK1 inhibitor, upadacitinib. Vitiligo is a common chronic autoimmune disorder that causes patches of skin to lose pigmentation when melanocytes are attacked and destroyed, causing the skin to turn a milky-white color. The global prevalence of vitiligo is approximately 0.5% to 2%. Early depigmentation typically occurs on the face and extremities. In 2011, an international consensus classified vitiligo into segmental vitiligo and non-segmental vitiligo (NSV; Ezzedine et al., Pigment Cell Melanoma Res. 2012, 25 (3):E1-13). The majority of patients (>90%) exhibit the non-segmental variant, in which white patches usually appear symmetrically on both sides of the body, such as on both hands or both knees, with occasional rapid loss of pigment including over a large area. Hair (scalp, eyebrow, eyelash, beard, and body) and mucous membranes may also whiten. NSV can occur at any age. Segmental vitiligo (SV), which differs in appearance and causality from NSV, is much less common. SV tends to affect areas of skin that are associated with dorsal roots from the spinal cord and is most often unilateral, more stable/static in course, and does not improve with topical therapies or UV light. The segmental subtype of vitiligo often begins at an early age, progressing for 6 to 12 months and then typically stopping.

Impairment or death of melanocytes is believed to be the direct cause of vitiligo. Melanocytes produce the pigment melanin, which colors the skin and provides protection from UV radiation. While several theories describe vitiligo pathogenesis, the exact etiology is unknown. The autoimmune theory hypothesizes that interferon (IFN)-y is an important cytokine expressed in lesional skin and is required for the recruitment of melanocyte-specific, autoreactive CD8+T cells to the skin via the chemokines CXCL9/10 and CXCR3 (Rodrigues et al., J Am Acad Dermatol. 2017, 77 (1): 1-13; Harris et al., J Invest Dermatol. 2012, 132 (7): 1869-76). Cytotoxic CD8+T cells are both necessary and sufficient for melanocyte destruction in the skin of vitiligo patients and therefore serve as the effector arm driving the autoimmunity of vitiligo.

Overall, the effect of vitiligo on quality-of-life impairment seems to be associated with the distribution of vitiligo lesions (e.g., face, genital area) and also by the extent of the disease, highlighting the unmet need of effective systemic therapies. There are few treatment options for vitiligo, and these have limited efficacy. Thus, there is a lack of options for patients with vitiligo, particularly those with severe manifestation of the disease, given the limited response to currently available topical and systemic therapies. In view of the lack of treatment options and the emotional, psychological, and other costs associated with the disease, it is desirable in the art to provide patients with safe, well tolerated, and efficacious therapies for treatment of vitiligo.

### A. Adult Patient

Accordingly, in one aspect is provided a method of treating vitiligo in an adult human patient, the method comprising orally administering once daily to the adult human patient 15 mg of upadacitinib.

In some embodiments, the adult patient has an age in a range from about 18 to about 65 years.

In some embodiments, the adult patient has non-segmental vitiligo (NSV).

In some embodiments, the NSV is stable.

In some embodiments, the NSV is active.

In some embodiments, the adult patient has, before initiating the treatment, a Facial Vitiligo Area Scoring Index (F-VASI) of ≥0.5.

In some embodiments, the adult patient has, before initiating the treatment, a Total Vitiligo Area Scoring Index (T-VASI)≥5 T-VASI.

In some embodiments, the adult patient has not received any immunomodulatory biologic therapy prior to initiating the treatment.

In some embodiments, the adult patient has not received any topical JAK inhibitor within 12 weeks of initiating treatment.

In some embodiments, the adult patient has leukotrichia in ≤33% of the affected area. In some embodiments, the adult patient achieves F-VASI 75 at 24 weeks.

In some embodiments, the adult patient achieves at 36 weeks one or more of T-VASI 50, T-VASI 75, T-VASI 90, F-VASI 50, F-VASI 90.

In some embodiments, the patient achieves T-VASI 50 at 48 weeks after the first daily administration.

In some embodiments, the patient achieves F-VASI 75 at 48 weeks after the first daily administration.

In some embodiments, the patient achieves T-VASI 50 at 52 weeks after the first daily administration.

In some embodiments, the patient achieves F-VASI 75 at 52 weeks after the first daily administration.

In some embodiments, the adult patient achieves an improvement in Global Impression of Change-Overall Vitiligo (PaGIC-V) at 36 weeks.

In some embodiments, the adult patient achieves a vitiligo noticeability scale (VNS) score of “A lot less noticeable (4)” or “No longer noticeable (5)” at 36 weeks.

In some embodiments, the adult patient achieves an improvement from baseline in the vitiligo extent score (VES) at 36 weeks.

In some embodiments, the adult patient achieves an improvement from baseline in the VitiQoL at 36 weeks.

In some embodiments, the adult patient achieves a Dermatology Life Quality Index (DLQI) total score of “0” or “1” at 36 weeks.

In some embodiments, the adult patient achieves an improvement from baseline in the Hospital Anxiety and Depression Scale (HADS) scores at 36 weeks.

In some embodiments, the adult patient achieves an improvement in time to onset of repigmentation relative to a patient which has not been treated with upadacitinib.

In some embodiments, the adult patient achieves an improvement in itching or burning sensation within 36 weeks of initiating the treatment.

In some embodiments, the adult patient achieves a Physician's Global Impression of Change-Vitiligo (PhGIC-V) score of “Much better (1)” or “A little better (2)” at 36 weeks. In some embodiments, the adult patient achieves a Patient's Global Impression of Change-Vitiligo (PaGIC-V) score of “Much better (1)” or “A little better (2)” at 36 weeks. In some embodiments, the adult patient achieves a Face-Physician Global Vitiligo Assessment (F-PhGVA) score of “No depigmentation (0)” or “Limited extent of depigmentation (1)” at 36 weeks. In some embodiments, the adult patient achieves a Total-Physician Global Vitiligo Assessment (T-PhGVA) score of “No depigmentation (0)” or “Limited extent of depigmentation (1)” at 36 weeks. In some embodiments, the adult patient achieves a Face-Patient Global Vitiligo Assessment (F-PaGVA) score of “No depigmentation (0)” or “Limited extent of depigmentation (1)” at 36 weeks. In some embodiments, the adult patient achieves a Total-Patient Global Vitiligo Assessment (T-PaGVA) score of “No depigmentation (0)” or “Limited extent of depigmentation (1)” at 36 weeks.

In some embodiments, the method halts the progression of vitiligo within 36 weeks of initiating the treatment.

In some embodiments, the method results in a reduction from baseline in one or more biomarkers of vitiligo within 36 weeks of initiating the treatment. In some embodiments, the biomarker is an inflammatory chemotactic protein or a T-cell activation marker. In some embodiments, the biomarker is CXCL9, CXCL10, IL12B, TNFSF9, or a combination thereof.

In some embodiments, the adult patient is concomitantly treated with exposure to ultraviolet light.

The disclosed method generally comprises orally administering the upadacitinib to the adult patient daily for a period of time. In some embodiments, the administration is continued at the same dose and dosing frequency over a treatment period. The duration of the treatment period may vary. For example, the treatment period may be at least one month, such as 3 months, 4 months, 6 months, 9 months, 1 year, 2 years, 5 years, 10 years, 20 years, 50 years, or more. In some embodiments, the treatment period is 24 weeks. In some embodiments, the treatment period is 36 weeks. In some embodiments, the treatment period is 52 weeks. In some embodiments, the treatment period is at least 24 weeks, or at least 36 weeks. In some embodiments, the treatment period is 48 weeks.

### B. Pediatric Patient

In some embodiments, the patient with vitiligo is a pediatric human patient (i.e., less than about 18 years of age). In some embodiments, the dose administered to a pediatric patient may differ from that of an adult human patient.

Accordingly, in another aspect is provided a method of treating vitiligo in a pediatric human patient. The method generally comprises administering upadacitinib to the pediatric patient as a stable oral pharmaceutical formulation or an extended-release tablet, with dosing based on body weight. The amount of upadacitinib administered, the oral dose form, and the frequency of dosing (e.g., once or twice daily) will vary based on the weight of the patient.

In some embodiments, the pediatric patient has an age of less than 18 years. In some embodiments, the pediatric patient has an age of less than 12 years. In some embodiments, the pediatric patient has an age of less than 6 years. In some embodiments, the pediatric patient has an age in a range from about 2 to less than about 6 years, in a range from about 6 to less than about 12 years, or in a range from about 12 to less than about 18 years. In some embodiments, the pediatric patient has an age in a range from about 2 to about 18 years, such as about 2, about 3, about 4, about 5, about 6, about 7, about 8, about 9, about 10, about 11, about 12, about 13, about 14, about 15, about 16, about 17, or about 18 years of age.

In some embodiments, the pediatric patient has a body weight of at least about 10 kg. In some embodiments, the pediatric patient has a body weight from about 10 kg to less than about 30 kg, such as from about 20 to less than about 20 kg, or from about 20 to less than about 30 kg. In some embodiments, the pediatric patient has a body weight 30 kg or more.

In some embodiments, the pediatric patient has a body weight in a range from about 10 to less than about 20 kg, the method comprising administering 3 mg of upadacitinib twice daily (3 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 3 mg dose is provided BID as about 3 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises upadacitinib at a concentration of about 0.5 mg/mL and the 3 mg dose is provided BID as about 6 mL of the about 0.5 mg/ml solution.

In some embodiments, the pediatric patient has a body weight in a range from about 10 to less than about 20 kg, the method comprising administering 6 mg of upadacitinib twice daily (6 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 6 mg dose is provided BID as about 6 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises upadacitinib at a concentration of about 0.5 mg/mL and the 6 mg dose is provided BID as about 12 mL of the about 0.5 mg/ml solution.

In some embodiments, the pediatric patient has a body weight in a range from about 20 to less than about 30 kg, the method comprising administering 4 mg of upadacitinib twice daily (4 mg BID) as an oral solution. In some embodiments, the pediatric patient has a body weight in a range from about 20 to less than about 30 kg, the method comprising administering 8 mg of upadacitinib twice daily (8 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 8 mg dose is provided BID as about 8 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises upadacitinib at a concentration of about 0.5 mg/mL and the 8 mg dose is provided BID as about 16 mL of the about 0.5 mg/mL solution.

In some embodiments, the pediatric patient has a body weight of about 30 kg or greater, the method comprising administering 6 mg of upadacitinib twice daily (6 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 6 mg dose is provided BID as about 6 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises upadacitinib at a concentration of about 0.5 mg/mL and the 6 mg dose is provided BID as about 12 mL of the about 0.5 mg/mL solution.

In some embodiments, the pediatric patient has a body weight of about 30 kg or greater, the method comprising administering 15 mg of upadacitinib once daily (15 mg QD) as an extended-release tablet.

In some embodiments, the pediatric patient has a body weight of about 30 kg or greater, the method comprising administering 8 mg of upadacitinib twice daily (8 mg BID) as an oral solution.

In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL.

In some embodiments, the oral solution comprises upadacitinib at a concentration of about 1 mg/mL, and the 8 mg dose is provided BID as about 8 mL of the about 1 mg/mL solution.

In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises upadacitinib at a concentration of about 0.5 mg/mL and the 8 mg dose is provided BID as about 16 mL of the about 0.5 mg/ml solution.

In some embodiments, the pediatric patient has a body weight of about 30 kg or greater, the method comprising administering 15 mg of upadacitinib once daily (15 mg QD) as an extended-release tablet.

The maximal concentration achieved (Cₘₐₓ) with the aforementioned dosing may vary depending on e.g., the dose, the weight of the patient, and the individual patient's metabolism of upadacitinib.

In some embodiments, when an immediate release oral solution as described herein is administered BID to the pediatric subject, a mean Cₘₐₓ for upadacitinib is achieved in a range from about 20 to about 160 ng/mL.

In some embodiments, when administered twice daily at a dose of 3 or 4 mg each (3 or 4 mg BID) to the pediatric subject, a mean Cₘₐₓ for upadacitinib is achieved in a range from about 25 to about 50 ng/ml, such as from about 25 to about 35, from about 25 to about 33, from about 25 to about 31, from about 25 to about 29, or from about 25 to about 27 ng/ml.

In some embodiments, when administered twice daily at a dose of 6 or 8 mg each (6 or 8 mg BID) to the pediatric subject, a mean Cₘₐₓ for upadacitinib is achieved in a range from about 40 to about 100 ng/mL, such as from about 40 to about 95, from about 40 to about 90, from about 40 to about 85, from about 40 to about 80, from about 40 to about 75, from about 40 to about 70, from about 40 to about 65, from about 40 to about 60, from about 40 to about 55, from about 40 to about 50, or from about 40 to about 45 ng/mL.

In some embodiments, when an extended-release 15 mg tablet as described herein is administered once daily (15 mg QD) to the pediatric subject, a mean Cₘₐₓ for upadacitinib is achieved in a range from about 45 to about 50 ng/ml, such as from about 45 to about 49, from about 45 to about 48, from about 45 to about 46, or from about 45 to about 46 ng/mL.

The mean 24-hour exposure achieved (AUC₀₋₂₄) with the aforementioned dosing may vary, depending on e.g., the dose, the weight of the patient, the fed vs. fasted condition, and the individual patient's metabolism of upadacitinib.

In some embodiments, when an immediate release oral solution as described herein is administered BID to the pediatric subject, a mean AUC₀₋₂₄ for upadacitinib is achieved in a range from about 200 to about 700 ng·h/mL.

In some embodiments, when administered twice daily at a dose of 3 or 4 mg each (3 or 4 mg BID) to the pediatric subject, a mean AUC₀₋₂₄ for upadacitinib is achieved in a range from about, such as from about 220 to about 270 ng·h/mL, such as from about 220 to about 265, from about 220 to about 260, from about 220 to about 255, from about 220 to about 250, from about 220 to about 245, from about 220 to about 240, from about 220 to about 235, from about 220 to about 230, or from about 220 to about 225 ng·h/mL.

In some embodiments, when administered twice daily at a dose of 6 or 8 mg each (6 or 8 mg BID) to the pediatric subject, a mean AUC₀₋₂₄ for upadacitinib is achieved in a range from about 340 to about 590 ng·h/mL, such as from about 340 to about 580, from about 340 to about 570, from about 340 to about 560, from about 340 to about 550, from about 340 to about 540, from about 340 to about 530, from about 340 to about 520, from about 340 to about 510, from about 340 to about 500, from about 340 to about 490, from about 340 to about 480, from about 340 to about 470, from about 340 to about 460, from about 340 to about 450, from about 340 to about 440, from about 340 to about 430, from about 340 to about 420, from about 340 to about 410, from about 340 to about 400, from about 340 to about 390, from about 340 to about 380, from about 340 to about 370, from about 340 to about 360, from about 340 to about 350, or from about 340 to about 345 ng·h/mL. In some embodiments, when administered twice daily at a dose of 6 or 8 mg each (6 or 8 mg BID) to the pediatric subject, a mean AUC₀₋₂₄ for upadacitinib is achieved in a range from about 570 to about 590 ng·h/mL, such as from about 570 to about 590, from about 570 to about 588, from about 570 to about 586, from about 570 to about 584, from about 570 to about 582, or from about 570 to about 580 ng·h/mL.

In some embodiments, when an extended-release 15 mg tablet as described herein is administered once daily (15 mg QD) to the pediatric subject, a mean AUC₀₋₂₄ for upadacitinib is achieved in a range from about 45 to about 50 ng·h/mL, such as from about 45 to about 49, about 46 to about 48, or from about 47 to about 48 ng·h/mL.

In some embodiments, the pediatric patient has non-segmental vitiligo (NSV).

In some embodiments, the NSV is stable.

In some embodiments, the NSV is active.

In some embodiments, the pediatric patient has, before initiating the treatment, a Facial Vitiligo Area Scoring Index (F-VASI) of ≥0.5.

In some embodiments, the pediatric patient has, before initiating the treatment, a Total Vitiligo Area Scoring Index (T-VASI)≥5.

In some embodiments, the pediatric patient has not received any immunomodulatory biologic therapy prior to initiating the treatment.

In some embodiments, the pediatric patient has not received any topical JAK inhibitor within 12 weeks of initiating treatment.

In some embodiments, the pediatric patient has leukotrichia in ≤33% of the affected areas.

In some embodiments, the pediatric patient achieves F-VASI 75 at 24 weeks after initiating the treatment.

In some embodiments, the pediatric patient achieves at 36 weeks after initiating the treatment one or more of a T-VASI 50, a T-VASI 75, a T-VASI 90, a F-VASI 50, or a F-VASI 90.

In some embodiments, the pediatric patient achieves F-VASI 75 at 36 weeks after initiating the treatment.

In some embodiments, the pediatric patient achieves T-VASI 50 at 36 weeks after initiating the treatment.

In some embodiments, the pediatric patient achieves at 48 weeks after initiating the treatment one or more of a T-VASI 50, a T-VASI 75, a T-VASI 90, a F-VASI 50, or a F-VASI 90.

In some embodiments, the pediatric patient achieves F-VASI 75 at 48 weeks after initiating the treatment.

In some embodiments, the pediatric patient achieves T-VASI 50 at 48 weeks after initiating the treatment.

In some embodiments, the pediatric patient achieves at 52 weeks after initiating the treatment one or more of a T-VASI 50, a T-VASI 75, a T-VASI 90, a F-VASI 50, or a F-VASI 90.

In some embodiments, the pediatric patient achieves F-VASI 75 at 52 weeks after initiating the treatment.

In some embodiments, the pediatric patient achieves T-VASI 50 at 52 weeks after initiating the treatment.

In some embodiments, the pediatric patient achieves an improvement in Global Impression of Change-Overall Vitiligo (PaGIC-V) at 36 weeks after initiating the treatment.

In some embodiments, the pediatric patient achieves a vitiligo noticeability scale (VNS) score of “A lot less noticeable (4)” or “No longer noticeable (5)” at 36 weeks after initiating the treatment.

In some embodiments, the pediatric patient achieves an improvement from baseline in the vitiligo extent score (VES) at 36 weeks after initiating the treatment.

In some embodiments, the pediatric patient achieves an improvement from baseline in the VitiQoL at 36 weeks after initiating the treatment.

In some embodiments, the pediatric patient achieves a Dermatology Life Quality Index (DLQI) total score of “0” or “1” at 36 weeks.

In some embodiments, the pediatric patient achieves an improvement from baseline in the Hospital Anxiety and Depression Scale (HADS) scores at 36 weeks after initiating the treatment.

In some embodiments, the pediatric patient achieves an improvement in time to onset of repigmentation relative to a patient which has not been treated with upadacitinib.

In some embodiments, the pediatric patient achieves an improvement in itching or burning sensation within 36 weeks of initiating the treatment.

In some embodiments, the pediatric patient achieves a Physician's Global Impression of Change-Vitiligo (PhGIC-V) score of “Much better (1)” or “A little better (2)” at 36 weeks after initiating the treatment. In some embodiments, the pediatric patient achieves a Patient's Global Impression of Change-Vitiligo (PaGIC-V) score of “Much better (1)” or “A little better (2)” at 36 weeks after initiating the treatment. In some embodiments, the pediatric patient achieves a Face-Physician Global Vitiligo Assessment (F-PhGVA) score of “No depigmentation (0)” or “Limited extent of depigmentation (1)” at 36 weeks after initiating the treatment. In some embodiments, the pediatric patient achieves a Total-Physician Global Vitiligo Assessment (T-PhGVA) score of “No depigmentation (0)” or “Limited extent of depigmentation (1)” at 36 weeks after initiating the treatment. In some embodiments, the pediatric patient achieves a Face-Patient Global Vitiligo Assessment (F-PaGVA) score of “No depigmentation (0)” or “Limited extent of depigmentation (1)” at 36 weeks after initiating the treatment. In some embodiments, the pediatric patient achieves a Total-Patient Global Vitiligo Assessment (T-PaGVA) score of “No depigmentation (0)” or “Limited extent of depigmentation (1)” at 36 weeks after initiating the treatment.

In some embodiments, the method halts the progression of vitiligo within 36 weeks of initiating the treatment.

In some embodiments, the method results in a reduction from baseline in one or more biomarkers of vitiligo within 36 weeks of initiating the treatment. In some embodiments, the biomarker is an inflammatory chemotactic protein or a T-cell activation marker. In some embodiments, the biomarker is CXCL9, CXCL10, IL12B, TNFSF9, or a combination thereof.

In some embodiments, the pediatric patient is concomitantly treated with exposure to ultraviolet light.

The disclosed method generally comprises orally administering the upadacitinib to the pediatric patient daily for a period of time. In some embodiments, the administration is continued at the same dose and dosing frequency over a treatment period. The duration of the treatment period may vary. For example, the treatment period may be at least one month, such as 3 months, 4 months, 6 months, 9 months, 1 year, 2 years, 5 years, 10 years, 20 years, 50 years, or more. In some embodiments, the treatment period is 24 weeks. In some embodiments, the treatment period is at least 24 weeks, or at least 36 weeks. In some embodiments, the treatment period is 36 weeks. In some embodiments, the treatment period is 48 weeks. In some embodiments, the treatment period is 52 weeks.

### III. Pharmaceutical Compositions and Routes of Administration

Upadacitinib can be administered to a human patient by itself or in pharmaceutical composition where it is mixed with biologically suitable carriers or excipient(s) at doses to treat or ameliorate a disease or condition as described herein. Mixtures of these compounds can also be administered to the patient as a simple mixture or in suitable formulated pharmaceutical compositions.

The pharmaceutical compositions of the present disclosure may be manufactured in a manner that is itself known, e.g., by means of conventional mixing, dissolving, granulating, dragee-making, levigating, emulsifying, encapsulating, entrapping or lyophilizing processes.

Pharmaceutical compositions for use in accordance with the present disclosure thus may be formulated in a conventional manner using one or more physiologically acceptable carriers comprising excipients and auxiliaries which facilitate processing of the active compounds into preparations which can be used pharmaceutically. Proper formulation is dependent upon the route of administration chosen.

In some embodiments, the pharmaceutical composition is a tablet dosage form. In some embodiments, tablet is a controlled-release formulation, such as an extended-release tablet dosage form (also referred to herein as a modified release or sustained release formulation). Examples of solid dosage forms comprising upadacitinib may be found in International Patent Application Publication No. WO2017/066775, which is hereby incorporated by reference in its entirety.

In some embodiments, the composition is a stable liquid pharmaceutical composition. In some embodiments, the stable liquid pharmaceutical composition is a stable oral solution. In some embodiments, the stable liquid pharmaceutical composition is a stable oral suspension. Suitable stable liquid pharmaceutical compositions comprise upadacitinib or a pharmaceutically acceptable salt or solid-state form thereof, along with excipients such as buffers, preservatives, sweeteners, flavoring agents, pH adjusting agents, solvents, and the like. In some embodiments, the stable liquid pharmaceutical composition is a stable oral pharmaceutical solution comprising upadacitinib, a buffer and/or pH adjusting agent, a preservative, a sweetener, and water. In some embodiments, the stable liquid pharmaceutical composition is a stable oral pharmaceutical suspension comprising upadacitinib, a buffer and/or pH adjusting agent, a preservative, a sweetener, and water.

The concentration of upadacitinib in the stable liquid pharmaceutical composition may vary. Due to the bitterness of upadacitinib, which is difficult to mask at higher concentrations for palatability (see, e.g., Example 5), it is generally present at about 1 mg/mL or less. In some embodiments, the stable pharmaceutical composition is an oral solution comprising: upadacitinib at a concentration in a range from about 0.3 mg/mL to about 1.2 mg/mL, such as from about 0.3 to about 0.7 mg/mL, or from about 0.8 to about 1.2 mg/mL. In some embodiments, the oral solution comprises upadacitinib at a concentration of about 1 mg/mL, such as from about 0.8, 0.9, or about 1.0, to about 1.1 or about 1.2 mg/mL. In some embodiments, the oral solution comprises upadacitinib at a concentration from about 0.9 mg/mL to about 1.1 mg/mL. In some embodiments, the oral solution comprises upadacitinib at a concentration of 1.0 mg/mL. In some embodiments, the oral solution comprises upadacitinib at a concentration of about 0.5 mg/mL, such as from about 0.3, 0.4, or about 0.5, to about 0.6 or about 0.7 mg/mL. In some embodiments, the oral solution comprises upadacitinib at a concentration from about 0.4 mg/mL to about 0.6 mg/mL. In some embodiments, the oral solution comprises upadacitinib at a concentration of 0.5 mg/mL.

In some embodiments, the oral solution comprises a pH adjusting agent. Suitable pH adjusting agents include acids such as mineral or organic acids. Mineral acids include but are not limited to hydrochloric, sulfuric, and phosphoric acid. As used herein, the term “organic acid” refers to an organic (i.e., carbon-based) compound that is characterized by acidic properties. Typically, organic acids are relatively weak acids (i.e., they do not dissociate completely in the presence of water), such as carboxylic acids (—CO₂H). In some embodiments, the pH adjusting agent is an organic acid. Suitable organic acids include, but are not limited to, benzoic acid, toluic acids, salicylic acid, benzenesulfonic acid, p-toluenesulfonic acid, 2-(4-isobutylphenyl) propanoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid (L), aspartic acid (L), alpha-methylbutyric acid, camphoric acid (+), camphor-10-sulfonic acid (+), cinnamic acid, citric acid, cyclamic acid, dodecylsulfuric acid, ethane-1,2-disulfonic acid, ethanesulfonic acid, fumaric acid, furoic acid, galactaric acid, gentisic acid, glucoheptonic acid, gluconic acid, glucuronic acid, glutamic acid, glutaric acid, glycerophosphoric acid, glycolic acid, hippuric acid, isobutyric acid, isovaleric acid, lactobionic acid, lauric acid, levulinic acid, malic acid, maleic acid, malonic acid, mandelic acid, methanesulfonic acid, naphthalene-1,5-disulfonic acid, naphthalene-2-sulfonic acid, oleic acid, palmitic acid, pamoic acid, phenylacetic acid, pyroglutamic acid, pyruvic acid, sebacic acid, stearic acid, tartaric acid, and undecylenic acid. In some embodiments, the pH adjusting agent is selected from the group consisting of citric acid, phosphoric acid, tartaric acid, succinic acid, formic acid, acetic acid, and combinations thereof. In some embodiments, the pH adjusting agent is citric acid.

In some embodiments, the stable liquid pharmaceutical composition comprises a buffer. Suitable buffers include, but are not limited to, citrate, phosphate, tartrate, succinate, glycinate, glycerophosphate, formate, and acetate. In some embodiments, the buffer is selected from the group consisting of citrate, phosphate, tartrate, succinate, formate, acetate, and combinations thereof. In some embodiments, the buffer is selected from the group consisting of citrate and phosphate. In some embodiments, the buffer is sodium citrate.

The quantity of pH adjusting agent and/or buffer may vary. Generally, the concentration of each is adjusted to provide a desired pH range of the resulting stable liquid pharmaceutical composition. In some embodiments, the stable liquid pharmaceutical composition has a pH in a range from about 2 to about 5, or from about 3 to about 4, or from about 2 to about 3, or from about 4 to about 5, or from about 2.0 to about 2.5, or from about 2.5 to about 3.0, or from about 3.0 to about 3.5, or from about 3.5 to about 4.0, or from about 4.0 to about 4.5, or from about 4.5 to about 5.0, or from about 3.0 to about 3.1, or from about 3.0 to about 3.2, or from about 3.0 to about 3.3, or from about 3.1 to about 3.2, or from about 3.1 to about 3.3, or from about 3.1 to about 3.4, or from about 3.1 to about 3.5, or from about 3.2 to about 3.3, or from about 3.2 to about 3.4, or from about 3.2 to about 3.5, or from about 3.3 to about 3.4, or from about 3.3 to about 3.5. In some embodiments, the stable liquid pharmaceutical composition has a pH of about 3.0, or about 3.1, or about 3.2.

In some embodiments, the stable liquid pharmaceutical composition comprises a preservative. Suitable preservatives include, but are not limited to, benzoic acid, sodium benzoate, benzyl alcohol, ascorbic acid, potassium sorbate, 4-hydroxybenzoic acid, 4-hydroxybenzoate, methyl paraben, propyl paraben, sodium metabisulfite, and combinations thereof. In some embodiments, the preservative is selected from the group consisting of sodium benzoate, benzoic acid, propyl paraben, sodium metabisulfite, potassium sorbate, hydroxyparabenzoic acid, hydroxyparabenzoate, and combinations thereof.

In some embodiments, the stable liquid pharmaceutical composition comprises a sweetener. The sweetener can be any sweetener or combination of sweeteners, in natural or artificial form, or as a combination of natural and artificial sweeteners. Examples of natural sweeteners include fructose, sucrose, glucose, maltose, mannose, galactose, lactose, stevia, honey, and the like. Examples of artificial sweeteners include sucralose, isomaltulose, maltodextrin, saccharin, aspartame, acesulfame K, neotame, and the like. In some embodiments, the sweetener comprises one or more sugar alcohols. Sugar alcohols are polyols derived from monosaccharides or disaccharides that have a partially or fully hydrogenated form. Sugar alcohols have, for example, about 4 to about 20 carbon atoms and include erythritol, arabitol, ribitol, isomalt, maltitol, dulcitol, iditol, mannitol, xylitol, lactitol, sorbitol, and combinations thereof (e.g., hydrogenated starch hydrolysates). In some embodiments, the sweetener is selected from the group consisting of sucralose, acesulfame potassium, sodium saccharin, neotame, sucrose, maltitol, xylitol, and combinations thereof.

In some embodiments, the stable oral pharmaceutical solution comprises one or more flavoring agents. Any flavorful or aromatic substance capable of altering the taste, fragrance, or both of the solution may be utilized. Flavoring agents may be natural or synthetic. Suitable flavoring agents include, but are not limited to, flavor packages imparting flavors such as cherry, orange, lemon, lime, bubblegum, grape, strawberry, mango, and the like.

In some embodiments, the stable oral pharmaceutical solution comprises a taste modifier. Suitable taste modifiers include, but are not limited to, salts such as sodium chloride, and monoammonium glycyrrhizinate to accentuate sweetness.

In some embodiments, the stable pharmaceutical composition is an oral solution comprising upadacitinib, citric acid, sodium citrate, sodium benzoate, sweetener, and water.

In some embodiments, the stable pharmaceutical composition comprises citric acid in an amount in a range from about 0.1 to about 1 mg/mL.

In some embodiments, the stable pharmaceutical composition comprises sodium citrate in an amount in a range from about 0.01 to about 1 mg/mL.

In some embodiments, the stable pharmaceutical composition comprises sodium benzoate in an amount in a range from about 0.01 to about 0.1 mg/mL.

In some embodiments, the stable pharmaceutical composition comprises a sweetener in an amount in a range from about 1 to about 50 mg/mL.

In particular embodiments, the oral solution has a formulation as provided in Table 1.

TABLE 1
Example formulation of upadacitinib oral solution

| Component | mg/mL |
|---|---|
| upadacitinib | 0.3-1.2 |
| citric acid, anhydrous | 0.15-0.35 |
| sodium citrate, dihydrate | 0.03-0.06 |
| sodium benzoate | 0.02-0.06 |
| sucralose | 7-11 |
| purified water | q.s to 1 mL |

### EXEMPLIFICATION

### Example 1: A Multicenter, Randomized, Double-Blind, Placebo-Controlled Dose-Ranging Study to Evaluate the Safety and Efficacy of Upadacitinib in Subjects with Non-Segmental Vitiligo

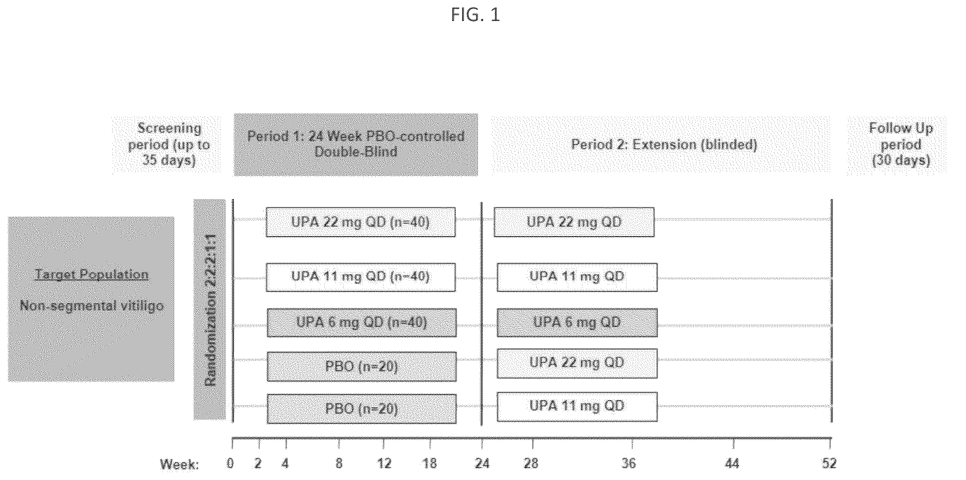

This study was a 52-week Phase 2, multiple-dose, placebo controlled double blind clinical trial in adult patients with non-segmental vitiligo. The study had two periods. The first period was a 24-week study and was followed by a blinded 28-week extension period which was blinded, but not placebo controlled. A schematic of the study is shown in FIG. 1.

The primary objective of this study was to evaluate the safety and efficacy of upadacitinib for the treatment of adult subjects with non-segmental vitiligo (NSV). The primary endpoint was the percent change from Baseline in Facial Vitiligo Area Scoring Index (F-VASI) at Week 24.

### Inclusion/Exclusion Criteria

Eligible subjects were adult females and males ≥18 to 65 years of age, inclusive, at Screening with a documented clinical diagnosis of NSV and no segmental or localized vitiligo. Subjects had either stable or active NSV. Approximately 130 adult male or female subjects were enrolled. Enrolled subjects met all of the following criteria at Screening and Baseline:
Facial Vitiligo Area Scoring Index (F-VASI)≥0.5 and total Vitiligo Area Scoring Index (T-VASI)≥5.
Subjects with other skin conditions that would interfere with evaluation of vitiligo, subjects with uncontrolled thyroid disease, and subjects with >33% leukotrichia on the face or >33% leukotrichia on the body (including face) were not eligible to enroll in this study.
Eligible subjects must not have previously been treated with any topical or systemic JAK inhibitor or permanent skin bleaching agents.
Subjects must not have been treated with any systemic vitiligo therapy (e.g., methotrexate, mycophenolate mofetil, corticosteroids), supplemental vitiligo therapy (antioxidants/vitamins/herbal medicine/traditional Chinese medicine), and/or topical vitiligo therapy including permanent or temporary tattoos within a minimum of 30 days prior to the first dose of study drug (Note: Camouflage and makeup may be used).
Eligible subjects must not have been treated with any phototherapy, including excimer (or other forms of laser therapy), within a minimum of 12 weeks prior to the first dose of study drug.
Subjects who had prior exposure to immunomodulatory biologic therapy, for any indications, must have discontinued the biologic therapy prior to the first dose of study drug. Recommended washout periods for biologic therapies include ≥4 weeks for etanercept; ≥8 weeks for adalimumab, infliximab, certolizumab, golimumab, abatacept, tocilizumab, and ixekizumab; ≥16 weeks for secukinumab; and ≥12 weeks for ustekinumab. For biologic therapies not specified, therapies must have been discontinued at least 5 times the mean terminal elimination half-life of a drug or 3 months prior to Baseline, whichever was longer.
Laboratory values meeting the following criteria within the Screening Period prior to the first dose of study drug were required:
   Serum aspartate transaminase (AST)≤2×upper limit of normal (ULN)
   Serum alanine transaminase (ALT)≤2×ULN
   Estimated glomerular filtration rate ≥30 mL/min/1.73 m² by simplified 4-variable Modification of Diet in Renal Disease (MDRD) formula
   Total WBC count ≥2,500/μL
   Absolute neutrophil count (ANC) ≥1,200/μL
   Platelet count≥100,000/μL
   Absolute lymphocyte count (ALC)≥750/μL
   Hemoglobin≥9 g/dL
Patients may not have had current or past history of the following:
   Two or more episodes of herpes zoster, or one or more episodes of disseminated herpes zoster
   One or more episodes of disseminated herpes simplex (including eczema herpeticum/Kaposi varicelliform eruption)
   Human immunodeficiency virus (HIV) infection, defined as confirmed positive anti-HIV antibody (HIV Ab) test
   Active tuberculosis (TB) or meet TB exclusionary parameters
   Positive result of beta-D-glucan (screening for pneumocystis jirovecii infection) or 2 consecutive indeterminate results of beta-D-glucan during the Screening Period (for subjects in Japan only)
   Active infection(s) requiring treatment with intravenous anti-infectives within 30 days, or oral/intramuscular (IM) anti-infectives within 14 days prior to the Baseline Visit
   Chronic recurring infection and/or active viral infection
   Confirmed COVID-19
   Hepatitis B virus (HBV): hepatitis B surface antigen positive (+) test or detectable HBV DNA PCR qualitative test for subjects who are hepatitis B core antibody (HBc Ab) positive (+) (and for hepatitis B surface antibody [HBs Ab] positive [+] subjects where mandated by local requirements)
   Hepatitis C virus (HCV): detectable HCV RNA in any subject with anti-HCV Ab.
   Recent (within past 6 months) cerebrovascular accident, myocardial infarction, coronary stenting, and aorto-coronary bypass surgery
   History of an organ transplant which requires continued immunosuppression
   History of GI perforation (other than due to appendicitis or mechanical injury), diverticulitis, or significantly increased risk for GI perforation per investigator judgment
   Conditions that could interfere with drug absorption including but not limited to short bowel syndrome or gastric bypass surgery; subjects with a history of gastric
   banding/segmentation are not excluded
   History of malignancy except for successfully treated NMSC or localized carcinoma in situ of the cervix
   Uncontrolled thyroid disease
   Other skin conditions that would interfere with evaluation of vitiligo
   >33% leukotrichia on the face or >33% leukotrichia on the body (including face)
Systemic use of known strong CYP3A inhibitors (including over-the-counter or prescription medicines, vitamins, and/or herbal supplements) was not permitted from Screening through the end of study drug administration and use of strong CYP3A inducers was not permitted from 30 days prior to study drug administration through the end of study drug administration. In addition, herbal therapies and other traditional medicines with unknown effects on CYP3A were not permitted from Screening through the end of study drug administration.

### Study Drug and Duration of Treatment

The study was comprised of a 35-day Screening Period, a 24-week double-blind treatment period (Period 1), a 28-week blinded long-term extension (Period 2), and a 30-day Follow-up Period. Subjects who meet eligibility criteria at Baseline were randomized in a 2:2:2:1:1 ratio to one of five treatment groups as follows:
Group 1: upadacitinib 22 mg once daily (QD) (N=40) (Period 1)→upadacitnib 22 mg QD (Period 2)
Group 2: upadacitinib 11 mg QD (N=40) (Period 1)→upadacitinib 11 mg QD (Period 2)
Group 3: upadacitinib 6 mg QD (N=40) (Period 1)→upadacitinib 6 mg QD (Period 2)
Group 4: placebo (N=20) (Period 1)→upadacitnib 22 mg bQD (Period 2)
Group 5: placebo (N=20) (Period 1)→upadacitnib 11 mg QD (Period 2)

The upadacitinib was administered orally once daily (QD).

### Stratification

Patients were randomized and stratified according to:
Age (less than or equal to 50; greater than 50)
Disease severity at baseline (T-VASI less than 15 or greater than/equal to 15)
Active vitiligo status (Yes/No)

### Study Objectives and Endpoints

### Efficacy Endpoints

The following efficacy parameters were used.

### Primary Endpoint:

Percent change from Baseline in F-VASI at Week 24 with upadacitinib treatment compared to placebo in the Intent-to-Treat (ITT) Population, which consisted of all randomized subjects.

### Secondary Endpoints:

Proportion of subjects achieving F-VASI 75, defined as at least a 75% improvement in F-VASI from Baseline at Week 24
Proportion of subjects achieving F-VASI 50, defined as at least a 50% improvement in F-VASI from Baseline at Week 24
Proportion of subjects achieving T-VASI 50, defined as at least a 50% improvement in T-VASI from Baseline at Week 24
Percent (%) change from Baseline in T-VASI at Week 24
Change from Baseline in the vitiligo quality-of-life (VitiQoL) instrument total score at Week 24

### Additional Endpoints

Achievement of F-VASI 90
Achievement of T-VASI 90
Achievement of T-VASI 75
Mean and percent change from Baseline in the vitiligo extent score (VES)
Achievement of vitiligo noticeability scale (VNS) score of “A lot less noticeable (4)” or “No longer noticeable (5)”
Dermatology Life Quality Index (DLQI) total score of “0” or “1”
Change from Baseline in the Hospital Anxiety and Depression Scale (HADS) scores
Proportion of subjects selecting each of the response categories of VitiQoL 16 questions
Achievement of Physician's Global Impression of Change-Vitiligo (PhGIC-V) of “Much better (1)” or “A little better (2)”
Achievement of Patient's Global Impression of Change-Vitiligo (PaGIC-V) of “Much better (1)” or “A little better (2)”
Achievement of Face-Physician Global Vitiligo Assessment (F-PhGVA) of “No depigmentation (0)” or “Limited extent of depigmentation (1)”
Achievement of Total-Physician Global Vitiligo Assessment (T-PhGVA) of “No depigmentation (0)” or “Limited extent of depigmentation (1)”
Achievement of Face-Patient Global Vitiligo Assessment (F-PaGVA) of “No depigmentation (0)” or “Limited extent of depigmentation (1)”
Achievement of Total-Patient Global Vitiligo Assessment (T-PaGVA) of “No depigmentation (0)” or “Limited extent of depigmentation (1)”

### Results

### Demographics

The study enrolled a high percentage of subjects with extensive vitiligo (T-VASI>10 BSA) and/or active vitiligo. The baseline demographics of the overall study population and of each group (placebo, 6 mg, 11 mg, 22 mg dose) is provided in Table 2. The baseline disease characteristics (e.g., active vs. stable, years since diagnosis, disease severity, etc.) are provided in Table 3.

TABLE 2
Patient demographics

| | | | Upadacitinib | | Upadacitinib | | Upadacitinib | | | |
|---|---|---|---|---|---|---|---|---|---|---|
| | PBO | | 6 mg qd | | 11 mg qd | | 22 mg qd | | TOTAL | |
| | n = 46 | | N = 49 | | N = 47 | | N = 43 | | N = 185 | |
| Female, n (%) | 29 | (63) | 26 | (53.1) | 34 | (72.3) | 26 | (60.5) | 115 | (62.2) |
| Age (yrs), mean (SD) | 46.8 | (10.5) | 45.1 | (11.7) | 45.5 | (11.9) | 48.2 | (11.1) | 46.3 | (11.3) |
| Age group ≤50 years, | 26 | (56.5) | 28 | (57.1) | 28 | (59.6) | 25 | (58.1) | 107 | (57.8) |
| n (%) | | | | | | | | | | |
| Race, n (%) | | | | | | | | | | |
| White | 32 | (76.2) | 32 | (71.1) | 33 | (75.0) | 33 | (78.6) | 130 | (75.1) |
| Black/African American | 4 | (9.5) | 3 | (6.7) | 3 | (6.8) | 1 | (2.4) | 11 | (6.4) |
| Asian | 6 | (14.3) | 6 | (13.3) | 7 | (15.9) | 6 | (14.3) | 25 | (14.5) |
| Hispanic, n (%) | 5 | (10.9) | 6 | (12.2) | 4 | (8.5) | 5 | (11.6) | 20 | (10.8) |
| BMI (kg/m²), mean (SD) | 27.3 | (8.1) | 27.6 | (5.7) | 27.5 | (6.6) | 27.9 | (5.8) | 27.6 | (6.6) |
| Fitzpatrick skin | | | | | | | | | | |
| types, n (%) | | | | | | | | | | |
| Type I | 3 | (6.5) | 1 | (2) | 1 | (2.1) | 1 | (2.3) | 6 | (3.2) |
| Type II | 11 | (23.9) | 13 | (26.5) | 15 | (31.9) | 14 | (32.6) | 53 | (28.6) |
| Type III | 17 | (37.0) | 17 | (34.7) | 19 | (40.4) | 17 | (39.5) | 70 | (37.8) |
| Type IV | 9 | (19.6) | 11 | (22.4) | 9 | (19.1) | 9 | (20.9) | 38 | (20.5) |
| Type V | 4 | (8.7) | 5 | (10.2) | 2 | (4.3) | 2 | (4.7) | 13 | (7.0) |

| | | | | | | | | | |
|---|---|---|---|---|---|---|---|---|---|
| Type VI | 2 | (4.3) | 2 | (4.1) | 1 | (2.1) | 0 | 5 | (2.7) |

TABLE 3
Baseline disease characteristics

| | Upadacitinib | Upadacitinib | Upadacitinib | |
|---|---|---|---|---|
| PBO | 6 mg qd | 11 mg qd | 22 mg qd | TOTAL |
| n = 46 | N = 49 | N = 47 | N = 43 | N = 185 |

Active vitiligo
status, n (%):

| | | | | | | | | | | |
|---|---|---|---|---|---|---|---|---|---|---|
| Active | 34 | (73.9) | 33 | (67.3) | 32 | (68.1) | 32 | (74.4) | 131 | (70.8) |
| Stable | 12 | (26.1) | 16 | (32.7) | 15 | (31.9) | 11 | (25.6) | 54 | (29.2) |
| Duration of vitiligo | 20.4 | (13.8) | 18.3 | (12.0) | 19.8 | (13.6) | 16.3 | (13.3) | 18.8 | (13.2) |
| disease since symptom | | | | | | | | | | |
| onset (yrs), mean (SD) | | | | | | | | | | |
| Duration of vitiligo | 18.5 | (13.8) | 16.0 | (11.4) | 17.9 | (13.9) | 14.7 | (13.7) | 16.8 | (13.2) |
| disease since diagnosis | | | | | | | | | | |
| (yrs), mean (SD) | | | | | | | | | | |
| F-VASI, mean (SD) | 1.0 | (0.6) | 1.2 | (0.8) | 1.0 | (0.6) | 1.2 | (0.7) | 1.1 | (0.7) |
| T-VASI, mean (SD) | 21.0 | (17.0) | 21.0 | (16.0) | 22.3 | (18.2) | 21.8 | (15.9) | 21.5 | (16.7) |
| VES, mean (SD) | 16.9 | (16.5) | 18.8 | (18.1) | 17.7 | (15.3) | 20.2 | (16.9) | 18.4 | (16.7) |
| VitiQoL score, mean (SD) | 42.4 | (21.9) | 39.5 | (22.7) | 40 | (22.6) | 45.9 | (22.6) | 41.8 | (22.4) |
| History of vitiligo- | | | | | | | | | | |
| related symptoms, n (%) | | | | | | | | | | |
| Increased sensitivity | 22 | (47.8) | 30 | (61.2) | 29 | (61.7) | 27 | (62.8) | 108 | (58.4) |
| to sun/sunburn | | | | | | | | | | |
| Itching within | 15 | (32.6) | 17 | (34.7) | 10 | (21.3) | 10 | (23.3) | 52 | (28.1) |
| vitiligo lesions | | | | | | | | | | |
| Pain within | 4 | (8.7) | 7 | (14.3) | 4 | (8.5) | 3 | (7.0) | 18 | (9.7) |
| vitiligo lesions | | | | | | | | | | |
| Prior topical therapy | 22 | (47.8) | 19 | (38.8) | 20 | (42.6) | 18 | (41.9) | 79 | (42.7) |
| (TCS/TCI), n (%) | | | | | | | | | | |
| Prior phototherapy, n (%) | 12 | (26.1) | 4 | (8.2) | 9 | (19.1) | 10 | (23.3) | 35 | (18.9) |

### Subject Disposition

The subject disposition in Period 1 and Period 2 is provided for each treatment group in Tables 4 and 5, respectively. With reference to Tables 4 and 5, the majority of randomized patients completed the study, with few discontinuations observed.

TABLE 4
Subject disposition- period 1

| | | | Upadacitinib | | Upadacitinib | | Upadacitinib | |
|---|---|---|---|---|---|---|---|---|
| | PBO | | 6 mg qd | | 11 mg qd | | 22 mg qd | |
| Randomized | N = 46 | | N = 49 | | N = 47 | | N = 43 | |
| Treated | 46 | (100) | 49 | (100) | 47 | (100) | 43 | (100) |
| Completed Study | 43 | (93.5) | 45 | (91.8) | 45 | (95.7) | 33 | (76.7) |
| Drug in Period 1 | | | | | | | | |
| Prematurely | 3 | (6.5) | 4 | (8.2) | 2 | (4.3) | 10 | (23.3) |
| Discontinued Study | | | | | | | | |
| Drug | | | | | | | | |
| Withdrawal from | 2 | (4.3) | 1 | (2.0) | 1 | (2.1) | 5^{c} | (11.6) |
| Treatment by Subject | | | | | | | | |

| | | | | | | |
|---|---|---|---|---|---|---|
| Adverse Event | 0 | 1 | (2.0) | 0 | 4^{d} | (9.3) |

| | | | | | | |
|---|---|---|---|---|---|---|
| Lost to Follow-up | 0 | 2 | (4.1) | 1 | (2.1) | 0 |

| | | | | | | |
|---|---|---|---|---|---|---|
| Progressive Disease | 1 | (2.2) | 0 | 0 | 1 | (2.3) |

| | | | | |
|---|---|---|---|---|
| Lack of Efficacy | 0 | 0 | 0 | 0 |
| COVID-19 Infection | 0 | 0 | 0 | 0 |

TABLE 5
Subject disposition- period 2

| | PBO/ | | PBO/ | | | | | | | |
|---|---|---|---|---|---|---|---|---|---|---|
| | Upadacitinib | | Upadacitinib | | Upadacitinib | | Upadacitinib | | Upadacitinib | |
| | 11 mg qd | | 22 mg qd | | 6 mg qd | | 11 mg qd | | 22 mg qd | |
| Entered | N = 20 | | N = 22 | | N = 45 | | N = 45 | | N = 33 | |
| Treated | 20 | (100) | 22 | (100) | 45 | (100) | 45 | (100) | 33 | (100) |
| Ongoing Study | 5 | (25) | 10 | (45.5) | 20 | (44.4) | 18 | (40) | 13 | (39.4) |
| Drug in Period 2 | | | | | | | | | | |
| Completed Study | 14 | (70) | 11 | (50) | 21 | (46.7) | 21 | (46.7) | 17 | (51.5) |
| Drug in Period 2 | | | | | | | | | | |
| Prematurely | 1 | (5) | 1 | (4.5) | 4 | (8.9) | 6 | (13.3) | 3 | (9.1) |
| Discontinued | | | | | | | | | | |
| Study Drug | | | | | | | | | | |

| | | | | | | | | |
|---|---|---|---|---|---|---|---|---|
| Lost to | 0 | 0 | 1 | (2.2) | 4 | (8.9) | 1 | (3) |
| Follow-up | | | | | | | | |

| | | | | | | | | |
|---|---|---|---|---|---|---|---|---|
| Withdrawal from | 0 | 1 | (4.5) | 2 | (4.4) | 1 | (2.2) | 0 |
| Treatment by | | | | | | | | |
| Subject | | | | | | | | |

| | | | | | | | | | |
|---|---|---|---|---|---|---|---|---|---|
| Adverse Event | 1 | (5) | 0 | 1 | (2.2) | 1 | (2.2) | 1 | (3) |

| | | | | | | |
|---|---|---|---|---|---|---|
| Progressive | 0 | 0 | 0 | 0 | 1 | (3) |
| Disease | | | | | | |

| | | | | | |
|---|---|---|---|---|---|
| Lack of | 0 | 0 | 0 | 0 | 0 |
| Efficacy | | | | | |
| COVID-19 | 0 | 0 | 0 | 0 | 0 |
| Infection | | | | | |

### Efficacy:

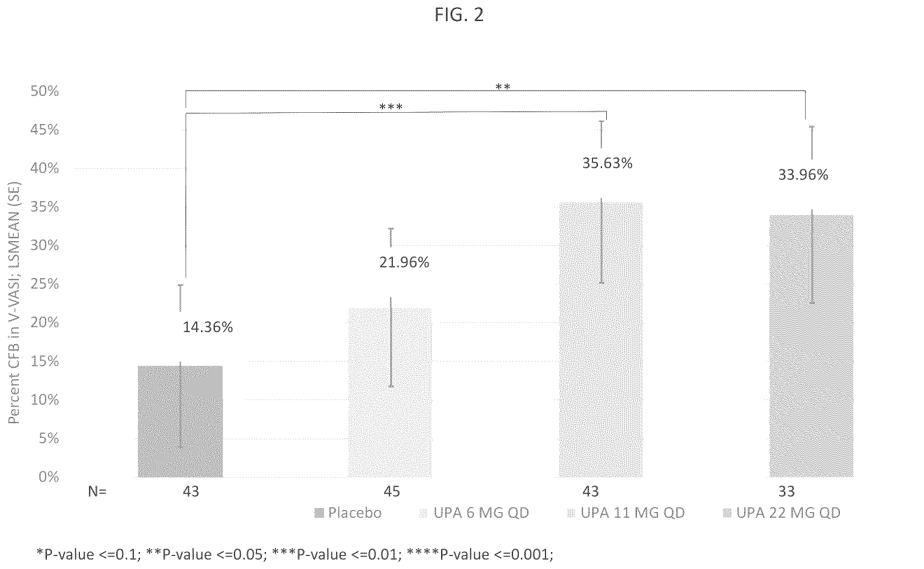

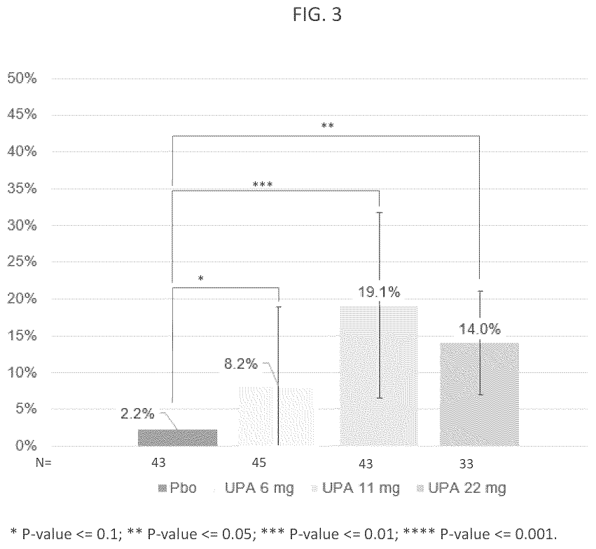

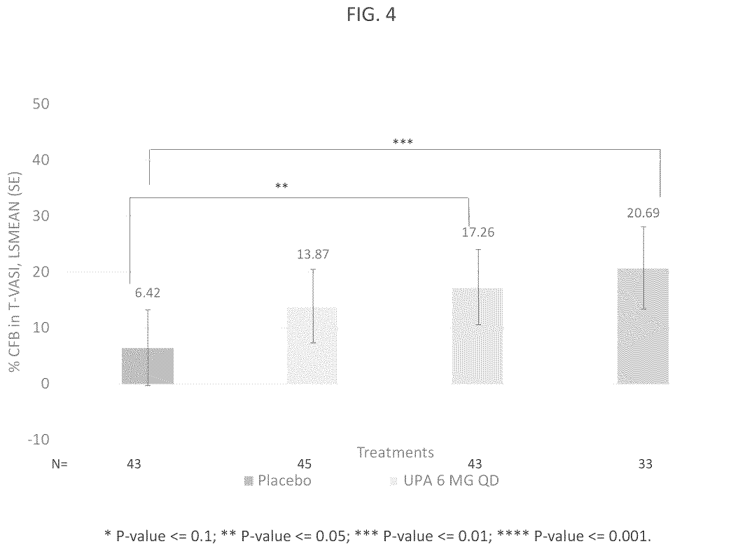

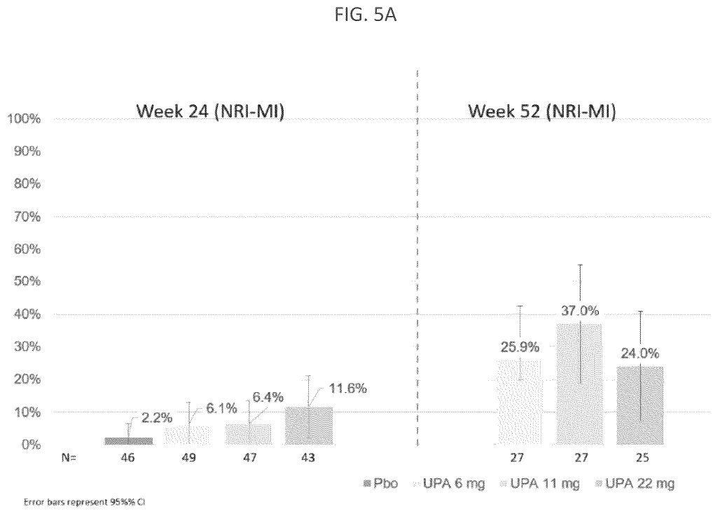

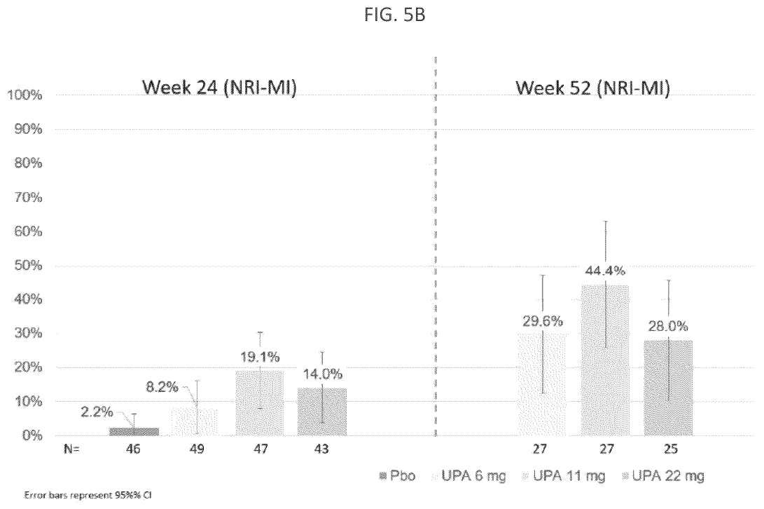

Overall, upadacitinib 11 mg and 22 mg met the primary endpoint (% change from baseline in F-VASI at Week 24), and also met most of the secondary endpoints (F-VASI 75, F-VASI 50, % change from baseline in T-VASI). In general, consistency of upadacitinib response was observed across pre-specified subgroups (Active and Stable, T-VASI≤10 and >10). The efficacy data are presented in FIGS. 2 to 5A/B and Tables 6 to 8. With reference to Table 6, upadacitinib 11 mg did not meet the T-VASI 50 secondary endpoint at week 24. Notably, the proportion of subjects achieving an F-VASI 75 and T-VASI 50 continued to increase through Week 52 with all doses of upadacitinib (for subjects having the opportunity to reach Week 52).

TABLE 6
Summary of proportion of patients meeting endpoints

| | | | | |
|---|---|---|---|---|
| Endpoints | | Upadacitinib | Upadacitinib | Upadacitinib |
| (All at | PBO | 6 MG QD | 11 MG QD | 22 MG QD |
| Week 24) | N = 46 | N = 49 | N = 47 | N = 43 |

Primary Endpoint:

| | | | | |
|---|---|---|---|---|
| Percent CFB | N = 43 | N = 45 | N = 43 | N = 33 |
| in F-VASI; | −14.36 (5.307) | −21.96 (5.162) | −35.63 (5.297) | −33.96 (5.793) |
| LSMEAN (SE) | | Δ = −7.60; | Δ = −21.27; | Δ = −19.60; |
| | | p = 0.304 | p = 0.005*** | p = 0.013** |

Secondary Endpoints:

| | | | | |
|---|---|---|---|---|
| T-VASI 50; | 1 (2.2) | 3 (6.1) | 3 (6.4) | 5 (11.6) |
| n (%) | | Δ = 3.7; | Δ = 3.8; | Δ = 9.1; |
| | | p = 0.340 | p = 0.358 | p = 0.027** |
| F-VASI 75; | 1 (2.2) | 4 (8.2) | 9 (19.1) | 6 (14.0) |
| n (%) | | Δ = 6.9; | Δ = 17.8; | Δ = 11.7; |
| | | p = 0.100* | p = 0.002*** | p = 0.026** |
| F-VASI 50; | 5 (10.9) | 8 (16.3) | 18 (38.3) | 17 (39.5) |
| n (%) | | Δ = 6.6; | Δ = 29.3; | Δ = 28.7; |
| | | p = 0.327 | p < 0.001**** | p < 0.001**** |
| Percent CFB | N = 43 | N = 45 | N = 43 | N = 33 |
| in T-VASI; | −6.42 (3.423) | −13.87 (3.332) | −17.26 (3.414) | −20.69 (3.731) |
| LSMEAN (SE) | | Δ = −7.45; | Δ = −10.84; | Δ = −14.27; |
| | | p = 0.120 | p = 0.026** | p = 0.005*** |
| CFB in | N = 40 | N = 44 | N = 44 | N = 34 |
| VitiQoL | −5.5 (2.41) | −7.5 (2.27) | −3.7 (2.30) | −6.6 (2.56) |
| Total Score; | | Δ = −1.9; | Δ = 1.9; | Δ = −1.1; |
| LSMEAN (SE) | | p = 0.545 | p = 0.565 | p = 0.754 |

With respect to Physician Global Impression of change (PhGIC) and Patient Global Impression of Change (PaGIC), patients achieved greater efficacy with upadacitinib 11 and 22 mg vs placebo (Table 7).

TABLE 7
Proportion of patients meeting additional endpoints

| Endpoints | | Upadacitinib | Upadacitinib | Upadacitinib |
|---|---|---|---|---|
| (All at | PBO | 6 MG QD | 11 MG QD | 22 MG QD |
| Week 24) | N = 46 | N = 49 | N = 47 | N = 43 |
| % Achieving | N = 46 | N = 49 | N = 47 | N = 43 |
| much better or | 11 (23.9%) | 21 (42.9%) | 28 (59.6%) | 24 (55.8%) |
| a little better | | Δ = 18% | Δ = 37.8% | Δ = 32% |
| on PhGIC, n (%) | | p = 0.043 | p < 0.001 | p < 0.001 |
| % Achieving | 9 (19.6%) | 17 (34.7%) | 26 (55.3%) | 26 (60.5%) |
| much better or | | Δ = 14.3% | Δ = 38.5% | Δ = 41.1% |
| a little better | | p = 0.081 | p < 0.001 | p = <0.001 |
| on PaGIC, n (%) | | | | |

TABLE 8
Patient Reported Outcomes at Week 24

| | Upadacitinib | | Upadacitinib | | Upadacitinib | | | |
|---|---|---|---|---|---|---|---|---|
| | 6 mg QD | | 11 mg QD | | 22 mg QD | | PBO | |
| | (N = 49) | | (N = 47) | | (N = 43) | | (N = 46) | |
| VitiQoL^{a} | | | | | | | | |
| LS mean change, % (SE) | −7.5 | (2.3) | −3.7 | (2.3) | −6.6 | (2.6) | −5.5 | (2.4) |

| | | | |
|---|---|---|---|
| P value^{b} | .545 | .565 | .754 |

VNS score of 4 or 5^{c}

| | | | | | |
|---|---|---|---|---|---|
| Responders, n (%) | 0 | 0 | 5 | (11.6) | 0 |

| | |
|---|---|
| P value^{b} | .005 |

DLQI^{d}

| | | | | | | | | |
|---|---|---|---|---|---|---|---|---|
| LS mean change, % (SE) | −1.4 | (0.5) | −1.2 | (0.5) | −2.2 | (0.5) | −0.6 | (0.5) |

| | | | |
|---|---|---|---|
| P value^{b} | .212 | .361 | .027 |

PaGIC-V score of 1 or 2^{e}

| | | | | | | | | |
|---|---|---|---|---|---|---|---|---|
| Responders, n (%) | 17 | (34.7) | 26 | (55.3) | 26 | (60.5) | 9 | (19.6) |

| | | | |
|---|---|---|---|
| P value^{b} | .081 | <.001 | <.001 |

HADS Anxiety^{f}

| | | | | | | | | |
|---|---|---|---|---|---|---|---|---|
| LS mean change, % (SE) | −0.7 | (0.4) | 0.0 | (0.4) | −0.3 | (0.4) | 0.3 | (0.4) |

| | | | |
|---|---|---|---|
| P value^{b} | .063 | .670 | .343 |

HADS Depression^{f}

| | | | | | | | | |
|---|---|---|---|---|---|---|---|---|
| LS mean change, % (SE) | 0.2 | (0.3) | −0.3 | (0.3) | −0.1 | (0.4) | 0.4 | (0.3) |

| | | | |
|---|---|---|---|
| P value^{b} | .638 | .150 | .352 |

DLQI, Dermatology Life Quality Index; HADS, Hospital Anxiety and Depression Scale; PaGIC-V, Patient's Global Impression of Change-Vitiligo; PBO, placebo; UPA, upadacitinib; VitiQoL, Vitiligo Quality of Life; VNS, Vitiligo Noticeability Scale.
^{a}VitiQoL change from baseline. Number of patients: UPA 6 mg, 44; UPA 11 mg 44; UPA 22 mg, 34; placebo 40.
^{b}P values are vs PBO and are nominal and not multiplicity adjusted.
^{c}VNS response was defined as a score of 4 (“a lot less noticeable”) or 5 (“no longer noticeable”).
^{d}DLQI change from baseline. Number of patients: UPA 6 mg, 43; UPA 11 mg 42; UPA 22 mg, 35; placebo 38.
^{e}PaGiC-V response was defined as a score of 1 (“much better”) or 2 (“a little better”).
^{f}HADS Anxiety score and HADS Depression score changes from baseline. Number of patients: UPA 6 mg, 44; UPA 11 mg 45; UPA 22 mg, 34; placebo 40.

Preliminary 3D imaging data support greater efficacy of upadacitinib 11 and 22 mg versus placebo (see Example 3). Specifically, patients receiving upadacitinib 11 and 22 mg demonstrated a greater percent change from baseline in vitiligo extent score (VES) by 3D imaging evaluation.

### Biomarkers

Reductions from baseline were observed in inflammatory chemotactic proteins (CXCL9 and 10) and T-cell activation markers (IL12B and TNFSF9), indicating an effect on type 1 inflammation (data not shown). The observed reductions in type 1 inflammation biomarkers suggest maximal inhibition was achieved at the 11 mg dose. Biomarker reductions were observed as early as week 2 and were maintained through week 24 with similar effects observed among all upadacitinib doses, although a somewhat lesser effect was observed in the 6 mg treatment group. For CXCL10, levels were reduced to those in healthy volunteers by week 2 in all dose groups. Overall, serum biomarker data support greater efficacy of UPA 11 mg and 22 mg vs placebo. No differences were apparent for active vs. stable subjects.

### Example 2: Analysis of Exposure-Response for Efficacy at Week 24

An analysis of drug exposure vs. response was performed using the data from Example 1. Regression models were developed for change from baseline in F-VASI, change from baseline in T-VASI, F-VASI 75 and T-VASI 50 at Week 24. Linear and non-linear regression models were evaluated for the continuous endpoints (change from baseline in F-VASI and T-VASI). A linear, logarithmic, treatment effect, and Emax model was evaluated for the categorical endpoints (F-VASI 75 and T-VASI 50). Stratification factors from the Phase 2 study were included in all the models evaluated. Final models for each endpoint were selected based on the Akaike Information Criterion and visual predictive checks.

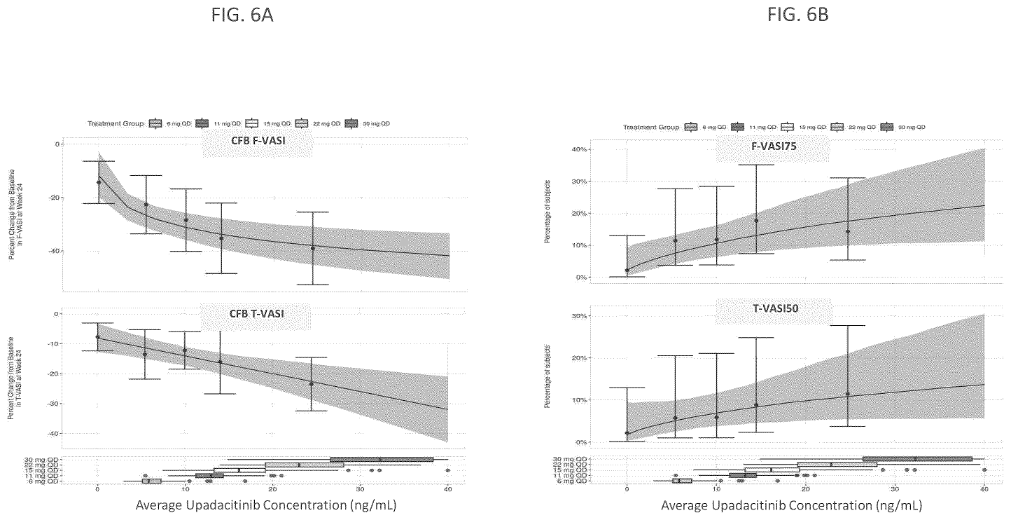

Preliminary exposure-response analyses for efficacy at Week 24, based on data from Period 1, showed clear exposure-response relationships for change from baseline in F-VASI, change from baseline in T-VASI, F-VASI 75, and T-VASI 50, with an increase in response with increasing upadacitinib average plasma exposures (C_{avg}; FIGS. 6A and 6B). These relationships were described using regression models for the continuous endpoints (change from baseline in F-VASI and T-VASI) and logistic regression models for the categorical endpoints (F-VASI 75 and T-VASI 50). A logarithmic model was selected for change from baseline in F-VASI, F-VASI 75, T-VASI 50, and a linear model was selected for change from baseline in T-VASI, where the exposure-response relationships for change from baseline in F-VASI, change from baseline in T-VASI, and F-VASI 75 were significant (P<0.05). The exposure-response relationship for T-VASI 50 was not significant, likely due to the limited number of responders for this endpoint.

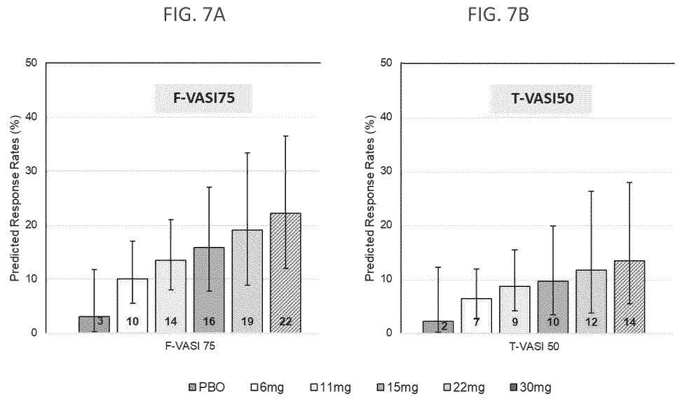

Simulations were conducted for 1000 trials with 400 subjects in each trial for placebo, 6 mg QD, 11 mg QD, 15 mg QD, 22 mg QD and 30 mg QD regimens and predicted response rates are provided in FIGS. 7A and 7B. With reference to FIG. 7A/B, simulations predict that 22 mg QD upadacitinib is predicted to provide 3 to 5% greater efficacy than that for the 11 mg dose for F- and T-VASI at week 24, and upadacitinib 15 mg or 30 mg QD are predicted to provide limited additional efficacy benefit relative to 11 mg or 22 mg QD upadacitinib, respectively.

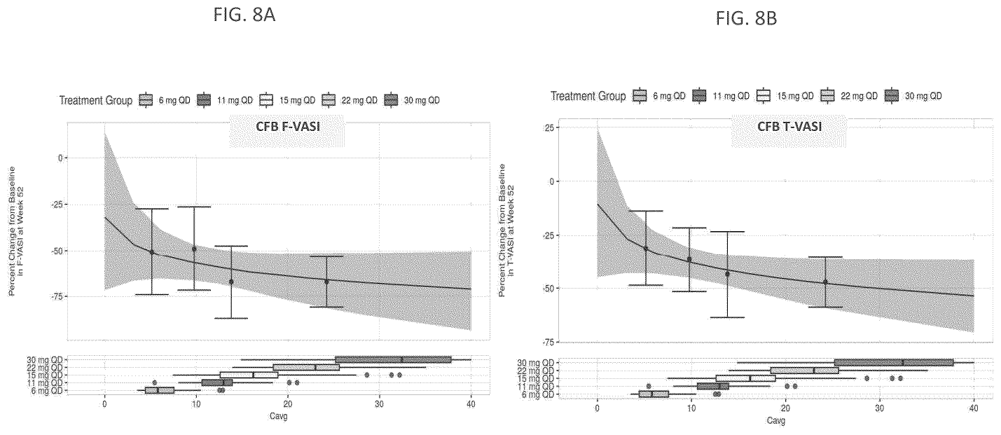

Preliminary exposure-response analyses for all the subjects that had completed Week 52 were conducted for change from baseline in F-VASI and change from baseline in T-VASI. Limited data were available for Week 52. A logarithmic model best described these exposure-response relationships at Week 36 and indicated that the efficacy appears to approach a plateau at plasma exposures associated with the 15 mg QD regimen (FIGS. 8A and 8B). The exposure-response relationships were shallower for change from Baseline in F-VASI and T-VASI at Week 52 compared to Week 24. Therefore, a dose higher than 15 mg is not predicted to provide meaningful additional efficacy benefit at Week 52.

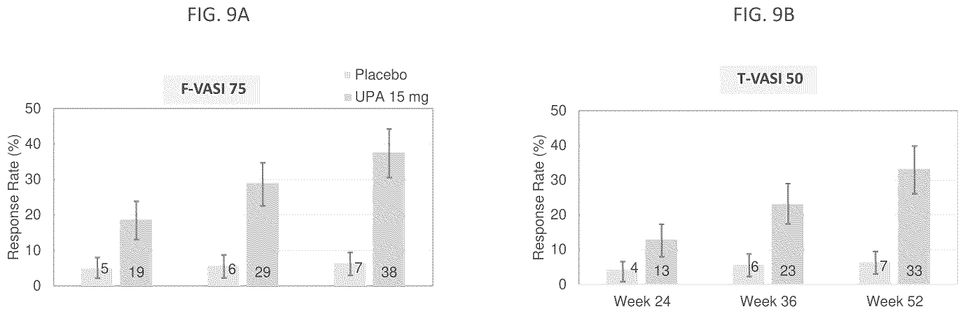

Based on all available data (pharmacokinetic, CFB T-VASI, and CFB F-VASI) at all available visits through Week 52, a longitudinal model was developed. The model-predicted efficacy was generally in agreement with the observed data over 52 weeks of treatment and shows the increase in treatment effect over time (FIGS. 9A and 9B). With reference to FIGS. 9A and 9B, the model-predicted effect sizes for upadacitinib 15 mg at Week 36 are estimated to be: F-VASI 75:23.2% (15.9% to 30.4%) and T-VASI 50:17.4% (10.9% to 23.9%).

Upadacitinib 15 mg QD is approved for the treatment of adults in RA, PsA, AD, AS, nr-axSpA, and UC (maintenance treatment) and in adolescents with AD. In addition, this dosing regimen has a robust and well-characterized safety profile across the different indications. The Phase 2b Study of Example 1 evaluated upadacitinib doses of 6 mg QD, 11 mg QD, and 22 mg QD in a placebo-controlled manner for 24 weeks. As detailed herein above, dose-response and biomarker results from this Phase 2b study demonstrate efficacy, safety, and pharmacodynamic response of 11 mg QD and higher doses in vitiligo.

As described above, exposure-response analyses of the Phase 2b study demonstrated that efficacy in vitiligo approaches plateau at plasma exposures associated with 15 mg QD. Although the proposed Phase 3 dosing regimen of 15 mg QD (Example 4) was not studied in the Phase 2 study of Example 1, it is estimated to provide plasma exposures that are within the range of plasma exposures that were evaluated in the Phase 2b study of Example 1.

### Example 3. Clinical Validation of 3D Digital Imaging

In the clinical study of Example 2, 3D imaging data was obtained and analyzed to aid in evaluating treatment efficacy. Specifically, multiple stereoscopic images were captured by a proprietary optical 3D scanner for a subset of patients in the referenced study. The data acquisition procedure and determination of vitiligo area using the 3D platform were as follows:
1. Subject was scanned, acquiring 2D raw color images with real time feedback to generate 3D gray low-resolution model
2. Full resolution color model generated and uploaded to cloud
3. Web application opened by investigator, who manually selected threshold for each region of face.
4. The detected vitiligo area was measured and results reported

Two objectives were predefined for the clinical validation of the 3D digital imaging output in vitiligo patients in the study: to determine the clinical validity of the 3D imaging platform in the vitiligo population by correlating facial 3D vitiligo measures with two established clinical measures of vitiligo, F-VASI, FPhGVA, and Patient Global Assessment of Vitiligo-Face; and to assess whether the vitiligo area measurement by 3D imaging platform can be utilized to determine a clinically meaningful change following upadacitinib therapy, supporting the 3D imaging platform-based facial vitiligo area measurement to determine upadacitinib efficacy in vitiligo patients.

To accomplish these objectives, twenty-seven subjects were enrolled into the 3D imaging substudy across 6 clinical sites. Twenty-one subjects had high quality 3D images available at Week 24 for analysis. Subjects who enrolled in the 3D digital imaging substudy were randomized as per the main protocol. The demographics, including Fitzpatrick skin types, and baseline disease characteristics in the 3D imaging substudy are provided in Table 9. With reference to Table 9, the demographics were in general consistent with the overall study population.

TABLE 9
Subject Demographics and Baseline Disease Characteristics in 3D Imaging Sub-study

| | | | Upadacitinib | | Upadacitinib | | Upadacitinib | | | |
|---|---|---|---|---|---|---|---|---|---|---|
| | PBO | | 6 mg QD | | 11 mg QD | | 22 mg QD | | TOTAL | |
| | N = 4 | | N = 9 | | N = 7 | | N = 7 | | N = 27 | |
| Female, n (%) | 2 | (50.0) | 5 | (55.6) | 5 | (71.4) | 4 | (57.1) | 16 | (59.3) |
| Age (years) mean (SD) | 50.3 | (7.32) | 43.7 | (11.7) | 49.1 | (11.42) | 39.4 | (13.45) | 45.0 | (12.45) |
| Age group ≤50 years, n (%) | 2 | (50.0) | 6 | (66.7) | 3 | (42.9) | 5 | (71.4) | 16 | (59.3) |

Race, n (%)

| White | 2 | (100) | 4 | (50.0) | 6 | (85.7) | 5 | (71.4) | 17 | (70.8) |
|---|---|---|---|---|---|---|---|---|---|---|
| Black/African American | 0 | | 0 | | 0 | | 0 | | 0 | |
| BMI (kg/m²), mean (SD) | 24.76 | (3.021) | 25.06 | (3.040) | 27.19 | (5.420) | 29.16 | (6.139) | 26.63 | (4.750) |

Fitzpatrick skin types, n (%)

| Type I | 0 | | 0 | | 0 | | 0 | | 0 | |
|---|---|---|---|---|---|---|---|---|---|---|
| Type II | 1 | (25.0) | 3 | (33.3) | 3 | (42.9) | 4 | (57.1) | 11 | (40.7) |
| Type III | 1 | (25.0) | 2 | (22.2) | 3 | (42.9) | 1 | (14.3) | 7 | (25.9) |
| Type IV | 2 | (50.0) | 2 | (22.2) | 1 | (14.3) | 2 | (28.6) | 7 | (25.9) |

| | | | | | | | |
|---|---|---|---|---|---|---|---|
| Type V | 0 | 2 | (22.2) | 0 | 0 | 2 | (7.4) |

| | | | | | |
|---|---|---|---|---|---|
| Type VI | 0 | 0 | 0 | 0 | 0 |

Active vitiligo status, n (%)

| Active | 3 | (75.0) | 9 | (100) | 4 | (57.1) | 5 | (71.4) | 21 | (77.8) |
|---|---|---|---|---|---|---|---|---|---|---|
| Stable | 1 | (25.0) | 0 | | 3 | (42.9) | 2 | (28.6) | 6 | (22.2) |
| Duration of vitiligo disease | 40.1 | (9.26) | 17.5 | (8.40) | 18.9 | (12.47) | 16.2 | (10.30) | 20.9 | (12.64) |
| since symptom onset (years), | | | | | | | | | | |
| mean (SD) | | | | | | | | | | |
| Duration of vitiligo disease | 34.5 | (12.44) | 16.4 | (8.38) | 17.2 | (11.84) | 15.9 | (10.66) | 19.1 | (11.87) |
| since diagnosis (years), mean | | | | | | | | | | |
| (SD) | | | | | | | | | | |
| F-VASI, mean (SD) | 1.313 | (0.7327) | 0.824 | (0.3796) | 0.924 | (0.5534) | 1.577 | (0.7472) | 1.118 | (0.6390) |
| T-VASI, mean (SD) | 12.545 | (4.5618) | 20.729 | (10.0861) | 17.066 | (15.1678) | 25.501 | (16.4254) | 19.804 | (12.9698) |

BMI = body mass index; F-VASI = Facial Vitiligo Area Scoring Index; PBO = placebo; QD = once daily; SD = standard deviation; TCI = topical calcineurin; TCS = topical corticosteroids; T-VASI = Total Vitiligo Area Scoring Index; UPA = upadacitinib; VitiQoL = vitiligo quality of life.

The quality and completeness of each scan was assessed by a visual inspection of the resulting 3D image by the technician and/or investigator, utilizing Wood's lamp visualization to set a threshold at Baseline, defined as the intensity value used to distinguish which 3D pixels within the image belong to the vitiligo area or to the normal skin area. The threshold value determined by the investigator at Baseline remained constant and applied to all post-baseline 3D images during the treatment period to reflect changes in the pigmentation. Each 3D image included 10 predefined anatomic regions of the face: forehead, left eye, right eye, left lateral cheek/temple, left middle cheek, nose, right middle cheek, right lateral cheek/temple, upper lip, and chin. Results are presented as the sum of vitiligo area measurement (cm²) across all anatomic regions of the face. A representative subject from the study at Baseline and Week 24 who was randomized to the upadacitinib 22 mg QD group is provided in Table 10. With reference to Table 10, the 3D imaging results of the total face show a percentage decrease in vitiligo area (−27.20%) close to what was observed for F-VASI (−24.62%).

TABLE 10
3D imaging versus F-VASI

| | | Cherry 3D imaging | | | | | |
|---|---|---|---|---|---|---|---|
| | Fiztpatrick | (Vitiligo area; cm²) | | | F-VASI | | |
| Treatment | Skin Type | Baseline | Wk 24 | % change | Baseline | Wk 24 | % change |
| Upadacitinib | Type III | 215.84 | 157.13 | −27.2 | 1.3 | 0.98 | −24.62 |
| 22 mg | (sometimes burns, | | | | | | |
| | tans moderately) | | | | | | |

Due to limited sites (N=6) involved in the 3D imaging substudy, investigators were only able to recruit 2 patients with Fitzpatrick skin types V and unable to recruit patients with Fitzpatrick skin types I and VI. In the study of Example 2, approximately 30% of subjects had Fitzpatrick skin types IV, V or VI. Once data from more patients are available, analyses are planned to determine if Fitzpatrick skin type has an impact on 3D imaging results and the correlation with other clinical measures.

Correlation of 3D imaging measurement to F-VASI was performed. A high correlation (R=0.865, P<0.001) between vitiligo area as measured by 3D imaging versus F-VASI at baseline was observed. Moderate correlation (R=0.56, P=0.007) in percent change from Baseline for 3D imaging versus percent change in F-VASI at Week 24 was observed. The trend in % change from baseline is reflected in both measurements with treatment. While good correlation between 3D imaging and F-VASI based on the % change from baseline to Week 24 was observed, some discrepancy was also noted. Specifically, although only 3 placebo subjects were included in the 3D imaging substudy, there was a large difference in the evaluation of placebo subjects between the 3D imaging versus F-VASI measurements. Individual values of the placebo subjects are presented in Table 11.

TABLE 11
Placebo Subjects in 3D Imaging Substudy

| | Vitiligo Area 3-D | | | | | |
|---|---|---|---|---|---|---|
| Fitzpatrick | Imaging (cm²) | | | F-VASI | | |
| Skin Type | Baseline | WK 24 | % CHG | Baseline | WK 24 | % CHG |
| Type II - | 123.03 | 89.81 | −27.00 | 2.2 | 1.98 | −10.00 |
| Burns | | | | | | |
| easily, | | | | | | |
| tans | | | | | | |
| minimally | | | | | | |
| Type IV - | 21.24 | 20.95 | −1.40 | 0.8 | 0.45 | −43.75 |
| Burns | | | | | | |
| minimally, | | | | | | |
| tans easily | | | | | | |
| Type IV - | 50.18 | 72.16 | 43.80 | 0.63 | 0.27 | −57.14 |
| Burns | | | | | | |
| minimally, | | | | | | |
| tans easily | | | | | | |

CHG = change; F-VASI = Facial Vitiligo Area Scoring Index; WK = Week.

A 3D image highlighting the vitiligo area in the left eye was evaluated and compared against F-VASI. The most different percentage change measured between the 3D imaging versus was: F-VASI, 3D: 43.80 versus F-VASI: −57.14 This finding suggests that 3D imaging not only provides visually verifiable images of the vitiligo but also offers better accuracy and objectivity over F-VASI,potentially reducing the placebo effect. A similar relationship (R=0.53, P<0.001) was observed in the moderate correlation of vitiligo area by 3D imaging versus the 5-point categorical F-PhGVA. All time points from baseline to Week 24 were included to ensure adequate samples for assessment of this categorical endpoint. Taken together, the correlations of 3D digital imaging with the established clinical assessments, F-VASI and F-PhGVA, at Baseline and post-treatment, support the clinical validity of 3D digital imaging technology for facial vitiligo assessment. The correlation of 3D imaging versus the 5-point categorical F-PaGVA resulted in a lower correlation (R=0.29, P=0.004), which was similar to weaker associations of patient assessments to VASI scores.

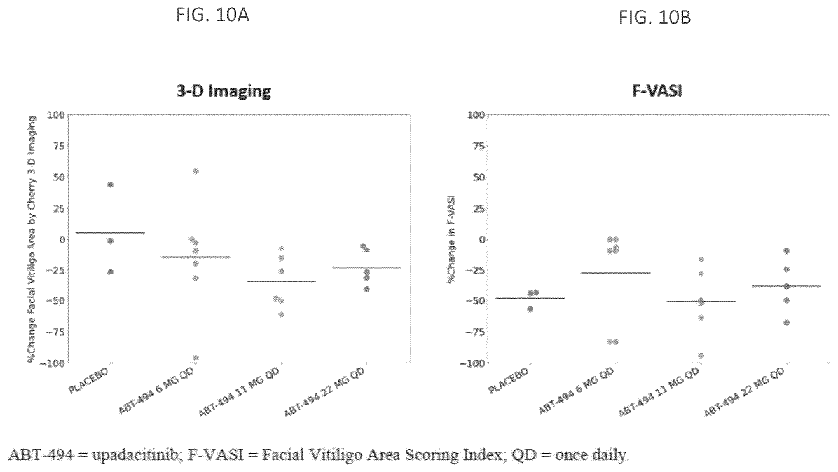

The Week 24 percent change from Baseline in the facial vitiligo area by 3D imaging is summarized in Table 12. At Week 24, the upadacitinib doses of 6 mg QD, 11 mg QD, and 22 mg QD resulted in percent reduction of facial vitiligo area from Baseline of 15.2%, 34.96%, and 23%, respectively, as measured by 3D imaging. The moderate to high correlation with F-VASI and the dose-response results, which are consistent with phase 2 primary analysis, suggest 3D can measure clinical meaningful change. The treatment effect size (Cohen's d) measured by 3D imaging versus F-VASI is compared and summarized in Table 13. Although the sample size was relatively small, the 3D imaging results show a strong effect size (>0.8) with the doses of 11 mg QD and 22 mg QD, which is larger than that of F-VASI. FIG. 10A provides percent change from Baseline in vitiligo area by 3D imaging by treatment group at Week 24 and FIG. 10B provides percent change in F-VASI by treatment group at Week 24 (individual patients and mean values (N=21)).

TABLE 12
Week 24 Percent Change from Baseline in
the Facial Vitiligo Area by 3D Imaging

| | % Change Facial Vitiligo Area by 3D Imaging at Week 24 | | | | | |
|---|---|---|---|---|---|---|
| Treatment | N | Mean (SD) | | Median | Min, Max | [95% CI] |
| Placebo | 3 | 5.14 | (35.85) | −1.38 | [−27.00, 43.80] | [−83.9, 94.2] |
| UPA 6 mg QD | 7 | −15.20 | (44.94) | −9.85 | [−96.02, 54.51] | [−56.8, 26.4] |
| UPA 11 mg QD | 6 | −34.96 | (21.52) | −37.38 | [−61.35, −7.66] | [−57.5, −12.4] |
| UPA 22 mg QD | 5 | −23.00 | (15.11) | −27.2 | [−40.97, −5.86] | [−41.8, −4.2] |

CI = confidence interval;
Max—maximum;
Min = minimum;
QD = once daily;
SD = standard deviation;
UPA = updacitinib

TABLE 13
Treatment Effect Size (Cohen's d) Measured
by 3D Imaging versus F-VASI at Week 24

| | Week 24 | | |
|---|---|---|---|
| | Placebo (N = 3) vs | Placebo (N = 3) vs | Placebo (N = 3) vs |
| | UPA 6 mg (N = 7) | UPA 11 mg (N = 6) | UPA 22 mg (N = 5) |
| Cohen's d of 3D | 0.50 | 1.36 | 1.02 |
| Imaging | | | |
| Cohen's d of F-VASI | 0.76 | 0.13 | 0.61 |

F-VASI = Facial Vitiligo Area Scoring Index;
UPA = upadacitinib

### Example 4: A 36-Week Placebo Controlled Double Blind Phase 3 Study

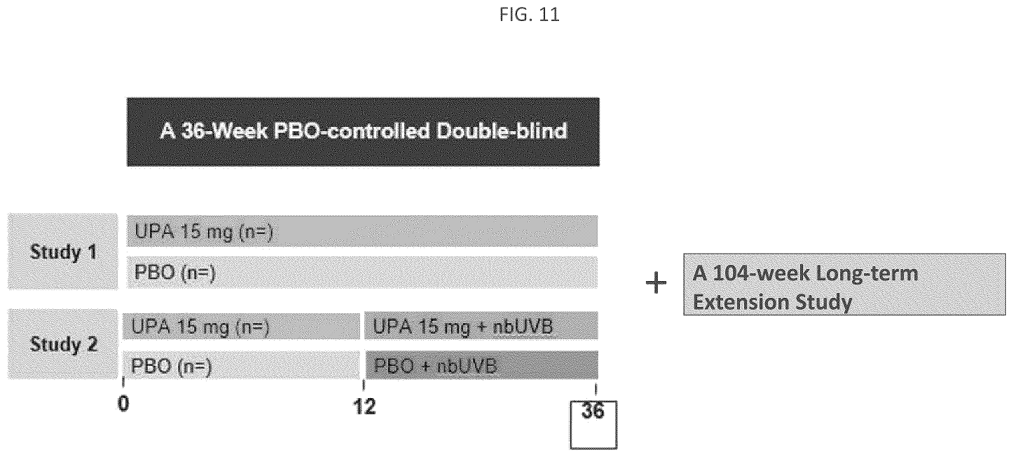

This is a 36-week Phase 3, single-dose, placebo controlled double blind clinical trial in adult patients with non-segmental vitiligo. The trial encompasses two studies. The first study is a 36-week study of 15 mg upadacitinib versus placebo. The second study is a 12-week study of 15 mg upadacitinib versus placebo, followed by ultraviolet (USB) phototherapy adjuvant treatment for 24 weeks (placebo versus 15 mg upadacitinib). Each of studies 1 and 2 will be followed by a 104-week extension period. A schematic of the study is shown in FIG. 11.

The selection of the 15 mg QD dose is based on the following:
The 11 mg QD and 22 mg QD regimens met the primary endpoint as well as most of the key secondary endpoints at Week 24, demonstrating higher efficacy of upadacitinib compared to placebo with nominal P-value<0.05. Both 11 mg and 22 mg QD regimens showed higher efficacy compared to placebo for the primary endpoint (percent change from baseline in F-VASI), F-VASI 50, F-VASI 75, and percent change from baseline in T-VASI. Only the 22 mg QD dosing regimen showed higher efficacy compared to placebo for T-VASI 50.
Biomarker data available from the Phase 2b study (Example 1) showed reductions in circulating type 1 inflammation biomarkers down to healthy volunteer levels, which suggests that maximal inhibition is achieved at the 11 mg dose. Biomarker reductions were observed as early as Week 2 and were maintained through Week 24. Similar effects were observed between the 11 mg and 22 mg dose levels, with a somewhat lesser reduction observed in the 6 mg treatment group.

### Inclusion/Exclusion Criteria

### For Study 1

Adult and adolescent (male and female)≥12 years of age
Leukotrichia in ≤33% of affected area
No segmental or localized vitiligo
No exposure to any topical JAK inhibitor for any indication within 12 weeks of Baseline Visit

### For Study 2

Adult only
Must not have lack of response to previous phototherapy, including narrow band UV-B (nbUVB), or equivalent laser therapy
Must not have any contraindication to nbUVB phototherapy:
   Photosensitive disorder, including but not limited to, SLE, dermatomyositis
   Use of photosensitizing medication
   Photo-induced epilepsy
   Cataracts, aphakia
   Genetic skin cancer syndrome, dysplastic nevus syndrome
   History of Treatment with Arsenic or Ionizing Radiation

### Efficacy Endpoints

### Primary:

Achievement of F-VASI 75 at Week 36

### Ranked Secondary (at Week 36)

Achievement of T-VASI 50
Achievement of F-VASI 50
Achievement of F-VASI 90
Patient-assessed Global Impression of Change-Overall Vitiligo (PaGIC-V)
vitiligo noticeability scale (VNS)
Halting progression at Week 12 (Only in the phototherapy Study)

### Additional Endpoints

vitiligo extent score (VES)
vitiligo quality of life (VitiQoL)
Hospital Anxiety and Depression Scale (HADS)
Dermatology Life Quality Index (DLQI)
Global Measures of Disease Severity
Halting progression (depigmentation)
Time to onset of repigmentation
Changes in itching or burning sensation
Skin color matching

### Example 5: Upadacitinib Immediate Release Liquid Formulation

For pediatric use, an oral solution was developed to improve acceptability (palatability and swallowability), stability, and manufacturability. Specifically, a stable oral pharmaceutical solution of upadacitinib at 1 mg/mL and 0.5 mg/mL were prepared. Upadacitinib has good solubility at low pH (shown in Table 14). Thus, low pH buffers such as citrate, phosphate, tartrate, and formate are suitable to prepare the oral solution. Buffers with higher pH ranges such as succinate and acetate are also suitable (refer to Example 6) but will result in an oral suspension. Citrate buffer was selected because it has favorable pKa (˜3.1), which is close to the final pH of the oral solution. Accordingly, a formulation was developed based on the solubility of upadacitinib in liquid with citric acid and sodium citrate added to completely dissolve upadacitinib.

Upadacitinib has strong bitterness above concentrations of 0.1 mg/mL. An acceptable and palatable formulation has a bitter intensity scale value below 1.0. Hence, a sweetener, taste masking or modifier agent, and flavoring agent can mitigate the bitter taste of upadacitinib. Sweeteners or combinations of sweeteners such as acesulfame potassium, sodium saccharin, sucralose, neotame, sucrose, maltitol, and xylitol are suitable for this oral solution. Taste modifiers such as sodium chloride, citric acid and monoammonium glycyrrhinizate are also suitable for this oral solution. Flavoring agents such as cherry, orange, bubblegum, strawberry, and mango can enhance the acceptability of the formulation.

Upadacitinib oral solution contains water and sweetener that are potential causes of microbial growth. Upadacitinib oral solution is also a multi-dose formulation. Hence preservative is added into the formulation to prevent microbial proliferation. Preservatives such as sodium benzoate and propyl paraben are suitable based on the final pH of the oral solution. Other preservatives such as sodium metabisulfite, benzoic acid, hydroxyparabenzoate, potassium sorbate and hydroxyparabenzoic acid can also be used based on the final pH of the oral solution or suspension.

The upadacitinib 1 mg/mL oral solution C contained citric acid, sodium citrate, sucralose, sodium benzoate, and water. The 1 mg/mL oral solution was clear and colorless to light yellow. The composition of certain 1 mg/mL and 0.5 mg/mL oral solutions is shown in Table 15.

TABLE 14
Solubility of upadacitinib in different aqueous media at 37° C.

| | pH | pH | Solubility |
|---|---|---|---|
| Medium | (Nominal) | (Final) | (mg/mL) |
| 0.1N HCl | 1.0 | 2.57 | 38.4 ± 1.5 |
| 50 mM phosphate | 2.01 | 3.08 | 10.5 ± 0.1 |
| 50 mM citrate | 3.00 | 3.39 | 4.48 ± 0.08 |
| 50 mM citrate | 4.01 | 4.16 | 1.00 ± 0.01 |
| 50 mM citrate | 5.03 | 5.01 | 0.289 ± 0.006 |
| 50 mM citrate | 6.01 | 5.96 | 0.196 ± 0.001 |
| 50 mM phosphate | 7.02 | 7.14 | 0.194 ± 0.001 |
| 50 mM phosphate | 8.02 | 7.99 | 0.200 ± 0.013 |
| 50 mM carbonate | 9.02 | 9.11 | 0.199 ± 0.006 |
| Water | 6.02 | 6.92 | 0.240 ± 0.004 |
| Fed State Simulated | 5.01 | 5.10 | 0.455 ± 0.006 |
| Intestinal Fluid (FeSSIF) | | | |
| FeSSIF | 6.50 | 6.58 | 0.262 ± 0.003 |

TABLE 15
Compositions of certain upadacitinib oral solutions

| | | Clinical A | Clinical B | C |
|---|---|---|---|---|
| Formulation | | 1 mg/mL | 0.5 mg/mL | 1 mg/mL |
| Component | Function | Quantity (g) | | |
| upadacitinib | Active | 0.100 | 0.050 | 0.100 |
| freebase | | | | |
| sodium benzoate | Preservative | 0.050 | 0.030 | 0.030 |
| citric acid, | pH modifier | 0.288 | 0.213 | 0.213 |
| anhydrous | | | | |
| sodium citrate, | Buffer | — | 0.116 | 0.045 |
| dihydrate | | | | |
| sucralose | Sweetener | 0.900 | 0.900 | 0.900 |
| purified water | Solvent | q.s. to 100 | q.s. to 100 | q.s. to 100 |

### Example 6. Upadacitinib Immediate Release or Extended-Release Liquid Formulation (Suspension)

An immediate release oral suspension is prepared to accommodate higher dosage strength or higher pH. The buffers, preservatives, and sweeteners listed in Example 4 can be applied in this formulation. Generally, an extended-release liquid formulation is prepared to provide extended release of upadacitinib for once-daily administration using a release rate modifier, such as an ion exchange resin. The liquid dosage forms comprise an upadacitinib-ion exchange resin complex. The upadacitinib-ion exchange resin complex comprises upadacitinib or a pharmaceutically acceptable salt thereof bound to an ion exchange resin. Suitable ion exchange resins include, but are not limited to, a sulfonated copolymer comprising styrene and divinylbenzene. In some such embodiments, the mobile, or exchangeable, cation is sodium. An exemplary cation ion exchange resin is AmberLite™ IRP 69 (DuPont).

### Example 7: Analysis of Efficacy Through Week 52

This Example provides efficacy data through week 52 for the clinical study of Example 1. As described above in Example 1, at week 24, upadacitinib met the primary endpoint of percent change from baseline in Facial Vitiligo Area Scoring Index (F-VASI) with 11 mg and 22 mg doses versus placebo in adults with non-segmental vitiligo (NSV). One-hundred sixty-six patients (166; 89.7%) continued to the 28-week blinded extension (period two) as described in Example 1. In period two, patients receiving upadacitinib during period one continued their respective regimens (upadacitinib 22 mg, N=33; upadacitinib 11 mg, N=45; upadacitinib 6 mg, N=45); patients who received placebo in period one were pre-assigned to receive either upadacitinib 11 mg (N=21) or upadacitinib 22 mg (N=22) in period 2.

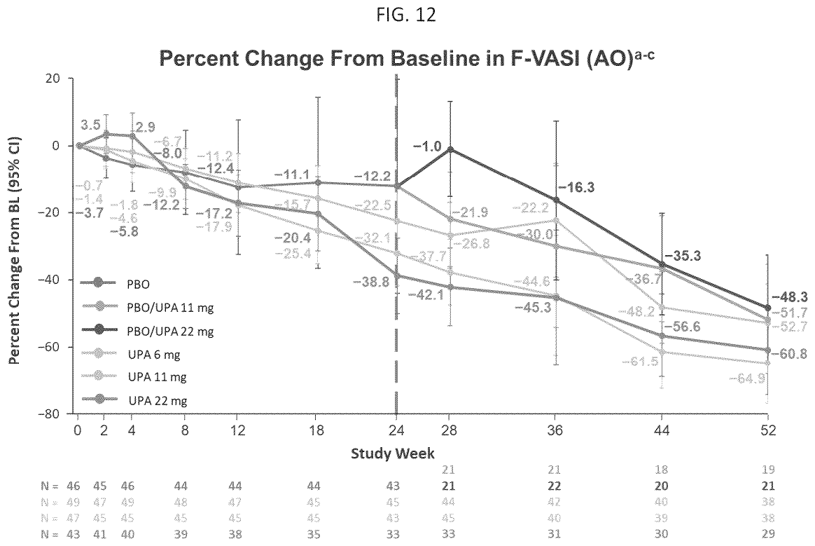

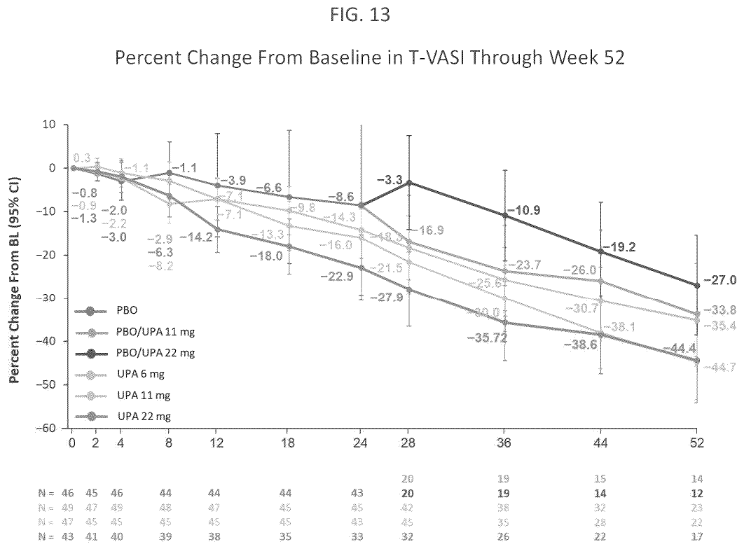

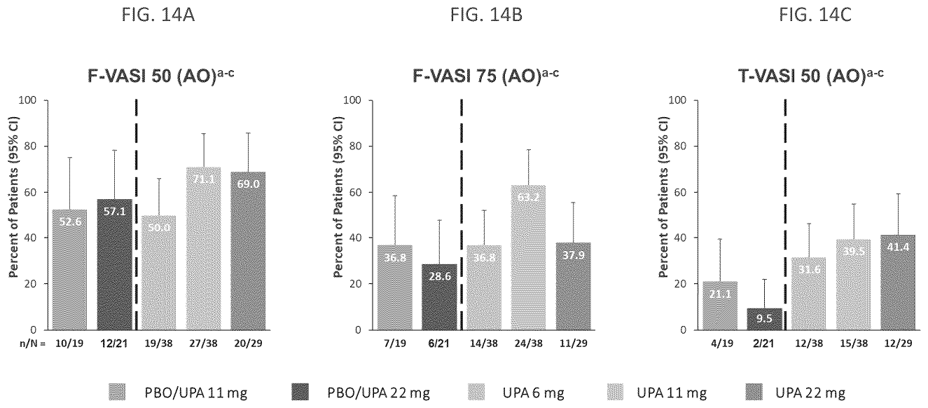

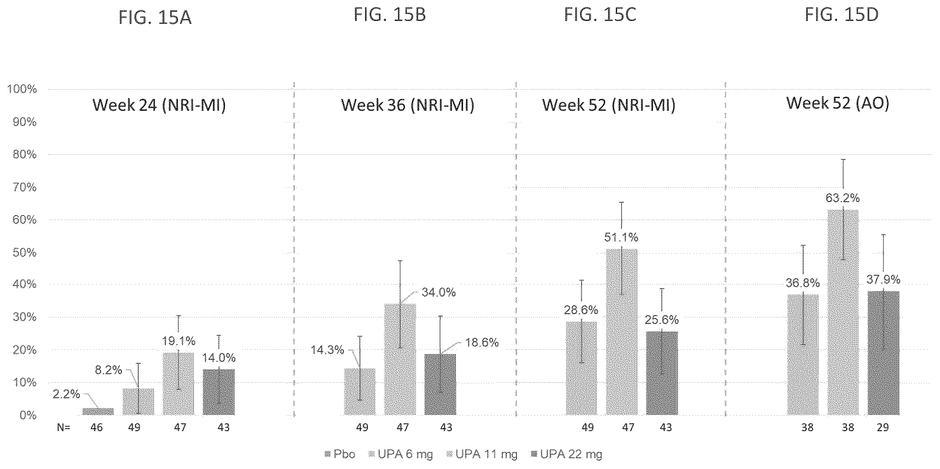

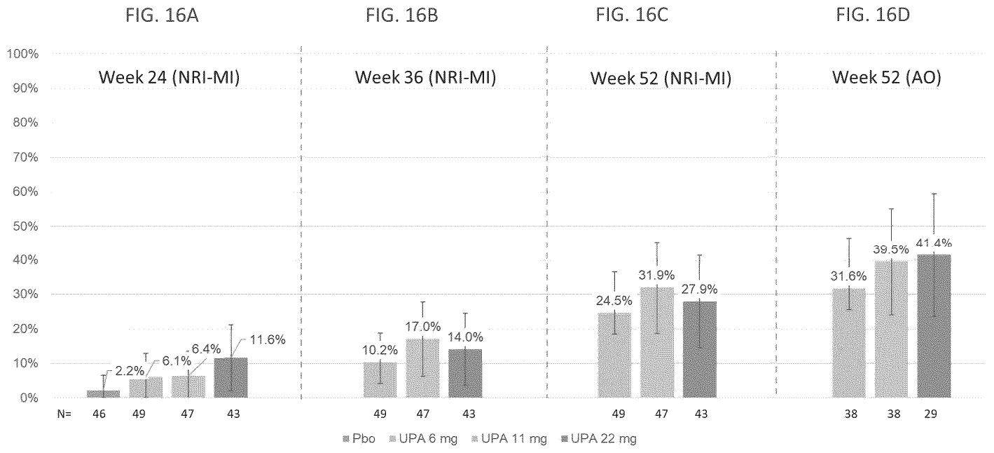

Data through week 52 are provided in Table 16 and Table 17 and FIG. 12 through FIG. 16D. With reference to Table 16, Table 17, and FIGS. 12-16, at week 52 of the study, the percent reduction from baseline in F-VASI was numerically greater than results observed at week 24 for all upadacitinib doses. In addition, response rates observed for F-VASI 75 and T-VASI 50 at week 52 were numerically greater than those at week 24 for all upadacitinib dose groups.

TABLE 16
Week 52 Data (Intent-to-Treat Population, MMRM/NRI-MI)

| | UPA 6 mg | UPA 11 mg | UPA 22 mg | PBO |
|---|---|---|---|---|
| | (N = 49) | (N = 47) | (N = 43) | (N = 46) |
| Percent CFB in | −52.3 | −62.8 | −59.2 | N/A |
| F-VASI | | | | |
| F-VASI 75 (%) | 28.6 | 51.1 | 25.6 | N/A |
| T-VASI 50 (%) | 24.5 | 31.9 | 27.9 | N/A |

TABLE 17
Week 52 Data (As Observed)

| | UPA 6 mg | UPA 11 mg | UPA 22 mg |
|---|---|---|---|
| | (N = 38) | (N = 38) | (N = 29) |
| Percent CFB in F-VASI | −52.7 | −64.8 | −60.8 |
| F-VASI 75 (%) | 36.8 | 63.2 | 37.9 |
| T-VASI 50 (%) | 31.6 | 39.5 | 41.4 |

What is claimed is:

1. A method of treating an adult human patient having vitiligo, the method comprising orally administering once daily to the patient 15 mg of upadacitinib.

2. The method of claim 1, wherein the patient has non-segmental vitiligo (NSV).

3. The method of claim 2, wherein the NSV is stable.

4. The method of claim 2, wherein the NSV is active.

5. The method of any one of claims 1-4, wherein the patient has, before initiating the treatment, a Facial Vitiligo Area Scoring Index (F-VASI) of ≥0.5.

6. The method of any one of claims 1-5, wherein the patient has, before initiating the treatment, a Total Vitiligo Area Scoring Index (T-VASI)≥5.

7. The method of any one of claims 1-6, wherein the patient has not received any immunomodulatory biologic therapy prior to initiating the treatment.

8. The method of any one of claims 1-7, wherein the patient has not received any topical JAK inhibitor within 12 weeks of initiating treatment.

9. The method of any one of claims 1-8, wherein the patient has leukotrichia in ≤33% of an affected area.

10. The method of any one of claims 1-9, wherein the patient achieves F-VASI 75 at 24 weeks after the first daily administration.

11. The method of any one of claims 1-10, wherein the patient achieves, at 36 weeks after the first daily administration, one or more of T-VASI 50, T-VASI 75, T-VASI 90, F-VASI 50, F-VASI 90.

12. The method of any one of claims 1-11, wherein the patient achieves an improvement in Global Impression of Change-Overall Vitiligo (PaGIC-V) at 36 weeks after the first daily administration.

13. The method of any one of claims 1-12, wherein the patient achieves a vitiligo noticeability scale (VNS) score of “A lot less noticeable (4)” or “No longer noticeable (5)” at 36 weeks after the first daily administration.

14. The method of any one of claims 1-13, wherein the patient achieves an improvement from baseline in the vitiligo extent score (VES) at 36 weeks after the first daily administration.

15. The method of any one of claims 1-14, wherein the patient achieves an improvement from baseline in the VitiQoL at 36 weeks after the first daily administration.

16. The method of any one of claims 1-15, wherein the patient achieves a Dermatology Life Quality Index (DLQI) total score of “0” or “1” at 36 weeks after the first daily administration.

17. The method of any one of claims 1-16, wherein the patient achieves an improvement from baseline in the Hospital Anxiety and Depression Scale (HADS) scores at 36 weeks after the first daily administration.

18. The method of any one of claims 1-17, wherein the patient achieves an improvement in time to onset of repigmentation relative to a patient which has not been treated with upadacitinib.

19. The method of any one of claims 1-18, wherein the patient achieves an improvement in itching or burning sensation within 36 weeks of initiating the treatment.

20. The method of any one of claims 1-19, wherein the patient achieves a Physician's Global Impression of Change-Vitiligo (PhGIC-V) score of “Much better (1)” or “A little better (2)” at 36 weeks after the first daily administration.

21. The method of any one of claims 1-20, wherein the patient achieves a Patient's Global Impression of Change-Vitiligo (PaGIC-V) score of “Much better (1)” or “A little better (2)” at 36 weeks after the first daily administration.

22. The method of any one of claims 1-21, wherein the patient achieves a Face-Physician Global Vitiligo Assessment (F-PhGVA) score of “No depigmentation (0)” or “Limited extent of depigmentation (1)” at 36 weeks after the first daily administration.

23. The method of any one of claims 1-21, wherein the patient achieves a Total-Physician Global Vitiligo Assessment (T-PhGVA) score of “No depigmentation (0)” or “Limited extent of depigmentation (1)” at 36 weeks after the first daily administration.

24. The method of any one of claims 1-23, wherein the patient achieves a Face-Patient Global Vitiligo Assessment (F-PaGVA) score of “No depigmentation (0)” or “Limited extent of depigmentation (1)” at 36 weeks after the first daily administration.

25. The method of any one of claims 1-24, wherein the patient achieves a Total-Patient Global Vitiligo Assessment (T-PaGVA) score of “No depigmentation (0)” or “Limited extent of depigmentation (1)” at 36 weeks after the first daily administration.

26. The method of any one of claims 1-25, wherein progression of vitiligo is halted within 36 weeks of initiating the treatment.

27. The method of any one of claims 1-26, wherein a reduction from baseline in one or more biomarkers of vitiligo is achieved within 36 weeks of initiating the treatment.

28. The method of claim 27, wherein the biomarker is an inflammatory chemotactic protein or a T-cell activation marker.

29. The method of claim 27 or 28, wherein the biomarker is CXCL9, CXCL10, IL12B, TNFSF9, or a combination thereof.

30. The method of any one of claims 1-29, wherein the method further comprises treating the patient concomitantly with exposure to ultraviolet light.

31. A method of treating vitiligo in a pediatric human patient, the method comprising administering a therapeutically effective amount of upadacitinib to the pediatric patient, wherein:
if the pediatric patient has a body weight in a range from about 10 kg to less than about 20 kg:
(i) the upadacitinib is administered twice daily at a dose of 3 mg each (3 mg BID), or
(ii) the upadacitinib is administered twice daily at a dose of 6 mg each (6 mg BID);
if the pediatric patient has a body weight in a range from about 20 kg to less than about 30 kg:
(i) the upadacitinib is administered twice daily at a dose of 4 mg each (4 mg BID), or
(ii) the upadacitinib is administered twice daily at a dose of 8 mg each (8 mg BID); and
if the pediatric patient has a body weight of about 30 kg or greater:
(i) the upadacitinib is administered twice daily at a dose of 6 mg each (6 mg BID),
(ii) the upadacitinib is administered twice daily at a dose of 8 mg each (8 mg BID), or
(ii) the upadacitinib is administered once daily at a dose of 15 mg (15 mg QD).

32. The method of claim 31, wherein the twice daily at a dose of 3 mg of upadacitinib, the twice daily at a dose of 4 mg of upadacitinib, the twice daily at a dose of 6 mg of upadacitinib or the twice daily at a dose of 8 mg of upadacitinib is administered to the pediatric patient as a stable oral pharmaceutical solution.

33. The method of claim 31 or 32, wherein the stable oral pharmaceutical solution comprises upadacitinib, a buffer and/or pH adjusting agent, a preservative, a sweetener, and water.

34. The method of any one of claims 31-33, wherein the oral solution comprises upadacitinib at a concentration of about 0.5 mg/mL or about 1 mg/mL.

35. The method of any one of claims 31-34, wherein the once daily at a dose of 15 mg of upadacitinib is administered to the pediatric patient as an extended-release tablet.

36. The method of any one of claims 31-35, wherein the pediatric patient has non-segmental vitiligo (NSV).

37. The method of claim 36, wherein the NSV is stable.

38. The method of claim 36, wherein the NSV is active.

39. The method of any one of claims 31-38, wherein the pediatric patient has, before initiating the treatment, a Facial Vitiligo Area Scoring Index F-VASI of ≥0.5.

40. The method of any one of claims 31-39, wherein the pediatric patient has, before initiating the treatment, a Total Vitiligo Area Scoring Index (T-VASI)≥5 T-VASI.

41. The method of any one of claims 31-40, wherein the pediatric patient has not received any immunomodulatory biologic therapy prior to initiating the treatment.

42. The method of any one of claims 31-41, wherein the pediatric patient has not received any topical JAK inhibitor within 12 weeks of initiating treatment.

43. The method of any one of claims 31-42, wherein the pediatric patient has leukotrichia in ≤33% of an affected area.

44. The method of any one of claims 31-43, wherein the pediatric patient achieves F-VASI 75 at 24 weeks after the first daily administration.

45. The method of any one of claims 31-44, wherein the pediatric patient achieves, at 36 weeks after the first daily administration, one or more of T-VASI 50, T-VASI 75, T-VASI 90, F-VASI 50, F-VASI 90.

46. The method of any one of claims 31-45, wherein the pediatric patient achieves an improvement in Global Impression of Change-Overall Vitiligo (PaGIC-V) at 36 weeks after the first daily administration.

47. The method of any one of claims 31-46, wherein the pediatric patient achieves a vitiligo noticeability scale (VNS) score of “A lot less noticeable (4)” or “No longer noticeable (5)” at 36 weeks after the first daily administration.

48. The method of any one of claims 31-47, wherein the pediatric patient achieves an improvement from baseline in the vitiligo extent score (VES) at 36 weeks after the first daily administration.

49. The method of any one of claims 31-48, wherein the pediatric patient achieves an improvement from baseline in the VitiQoL at 36 weeks after the first daily administration.

50. The method of any one of claims 31-49, wherein the pediatric patient achieves a Dermatology Life Quality Index (DLQI) total score of “0” or “1” at 36 weeks after the first daily administration.

51. The method of any one of claims 31-50, wherein the pediatric patient achieves an improvement from baseline in the Hospital Anxiety and Depression Scale (HADS) scores at 36 weeks after the first daily administration.

52. The method of any one of claims 31-51, wherein the pediatric patient achieves an improvement in time to onset of repigmentation relative to a patient which has not been treated with upadacitinib.

53. The method of any one of claims 31-52, wherein the pediatric patient achieves an improvement in itching or burning sensation within 36 weeks of initiating the treatment.

54. The method of any one of claims 31-53, wherein the pediatric patient achieves a Physician's Global Impression of Change-Vitiligo (PhGIC-V) score of “Much better (1)” or “A little better (2)” at 36 weeks after the first daily administration.

55. The method of any one of claims 1-54, wherein the pediatric patient achieves a Patient's Global Impression of Change-Vitiligo (PaGIC-V) score of “Much better (1)” or “A little better (2)” at 36 weeks after the first daily administration.

56. The method of any one of claims 1-55, wherein the pediatric patient achieves a Face-Physician Global Vitiligo Assessment (F-PhGVA) score of “No depigmentation (0)” or “Limited extent of depigmentation (1)” at 36 weeks after the first daily administration.

57. The method of any one of claims 1-56, wherein the pediatric patient achieves a Total-Physician Global Vitiligo Assessment (T-PhGVA) score of “No depigmentation (0)” or “Limited extent of depigmentation (1)” at 36 weeks after the first daily administration.

58. The method of any one of claims 1-57, wherein the pediatric patient achieves a Face-Patient Global Vitiligo Assessment (F-PaGVA) score of “No depigmentation (0)” or “Limited extent of depigmentation (1)” at 36 weeks after the first daily administration.

59. The method of any one of claims 31-58, wherein the pediatric patient achieves a Total-Patient Global Vitiligo Assessment (T-PaGVA) score of “No depigmentation (0)” or “Limited extent of depigmentation (1)” at 36 weeks after the first daily administration.

60. The method of any one of claims 31-59, wherein progression of vitiligo is halted within 36 weeks of initiating the treatment.

61. The method of any one of claims 31-60, wherein a reduction from baseline in one or more biomarkers of vitiligo is achieved within 36 weeks of initiating the treatment.

62. The method of claim 61, wherein the biomarker is an inflammatory chemotactic protein or a T-cell activation marker.

63. The method of claim 61 or 62, wherein the biomarker is CXCL9, CXCL10, IL12B, TNFSF9, or a combination thereof.

64. The method of any one of claims 21-63, wherein the method further comprises treating the pediatric patient concomitantly with exposure to ultraviolet light.

65. The method of any one of claims 1-9, wherein the patient achieves T-VASI 50 at 48 weeks after the first daily administration.

66. The method of any one of claims 1-9, wherein the patient achieves F-VASI 75 at 48 weeks after the first daily administration.

67. The method of any one of claims 1-9, wherein the patient achieves T-VASI 50 at 52 weeks after the first daily administration.

68. The method of any one of claims 1-9, wherein the patient achieves F-VASI 75 at 52 weeks after the first daily administration.
